(19) United States
(12) Patent Application Publication
(10) Pub. No.: US 2011/0237509 A1
(43) Pub. Date: Sep. 29, 2011

(54) hGH AND METHODS FOR PREPARING hGH

(75) Inventors: Matthew M. Skinner, Trafalgar, IN (US); Ronald Zimmerman, Greenwood, IN (US)

(73) Assignee: Elona Biotechnologies, Greenwood, IN (US)

(21) Appl. No.: 13/029,961

(22) Filed: Feb. 17, 2011

Related U.S. Application Data
(60) Provisional application No. 61/305,451, filed on Feb. 17, 2010.

(51) Int. Cl. A61K 38/27; C12N 15/18; C07K 14/61; C12N 1/21; C12N 15/63; A61P 5/00

(52) U.S. Cl. 514/11.4; 435/69.4; 530/399; 435/252.33; 435/320.1

(57) ABSTRACT

Preparations of recombinant human growth hormone (hGH) are provided having a purity that comprises 2% or less of a peptide other than native human growth hormone (e.g., essentially free of multimeric forms) are disclosed. Active pharmaceutical ingredient (API) preparations of recombinant hGH suitable for commercial production of formulation grade recombinant hGH are provided. A stable Master Cell Bank of transformed E. coli is also disclosed, as well as methods for creating a transformed E. coli preparation that includes a Met-AsphGH encoding sequence. Improved manufacturing methods are also presented that are suitable for large scale production of highly purified preparations of recombinant human growth hormone.

### PRIORITY CLAIM

The present application claims priority to U.S. Provisional Patent Application 61/305,451, filed Feb. 17, 2010.

### SEQUENCE LISTING

The instant application contains a Sequence Listing which has been submitted in ASCII format via EFS-Web and is hereby incorporated by reference in its entirety. Said ASCII copy, crated Feb. 17, 2011, is named 34344524.txt and is 25,114 bytes in size.

### BACKGROUND

1. Field of the Invention

The present invention relates to the field of methods of manufacturing human growth hormone, as an improved method for manufacturing recombinant human growth hormone is provided. In addition, the present invention relates to preparations of human growth hormone, as improved preparations of recombinant human growth hormone in active pharmaceutical ingredient (API) and formulation forms are provided.

2. Related Art

Growth hormone (GH) or somatotropin (STH) is a protein hormone which stimulates growth and cell reproduction in humans and other animals. Growth hormone is a polypeptide hormone synthesized in and secreted by the adenohypophysis (anterior lobe of the pituitary). Growth hormone is synthesized as a precursor protein (pre-growth hormone) containing an N-terminal signal peptide and the growth hormone sequence. Initial identification, purification and synthesis of growth hormone is associated with Choh Hao Li. Several diseases have now been characterized that are linked to GH deficiency and GH excess (acromegaly and pituitary gigantism). Initial uses of hGH included use in the treatment of Creutzfeldt-Jakob Disease and in the treatment of children's growth disorders. Different uses of human growth hormone continue to be identified, making this hormone of great clinical and commercial interest in human treatment.

The human form of growth hormone, hGH, is a protein that has a length of 191 amino acids, and has a molecular weight of about 21,124 kDa. hGH can be obtained from tissue, such as by extraction from pituitary glands, or may be produced by recombinant means. Two hGH forms that are obtained recombinantly are the 191 amino acid native species (Somatropin®), and the 192 amino acid N-terminal methionine (met) species (Somatrem®). Variants of hGH sequences, applications and production procedures are known; see for example U.S. Pat. Nos. 4,658,021, 4,665,160, 5,068,317, 5,079,345, 5, 424,199, 5,534,617, 5,597,709, 5,612,315, 5,633,352, 5,635,604, 5,688,666 and references cited therein.

Synthetic growth hormones available in the United States (and their manufacturers) include Nutropin® (Genentech), Humatrope® (Lilly), Genotropin® (Pfizer), Norditropin® (Novo), Tev-Tropin® (Teva) and Saizen® (Merck Serono). These products are known to vary in, among other ways, the formulations and delivery devices. A U.S. Food and Drug Association (FDA) approved follow-on version of rhGH is Onmitrope® (Sandoz). In addition, a sustained-release form of growth hormone, Nutropin® Depot (Genentech and Alkermes) has also been approved by the FDA. This formulation permits fewer injections (every 2 or 4 weeks instead of daily). However, this product is no longer available.

Like most large proteins, growth hormone readily undergoes various chemical and physical instability reactions. The predominant degradative reactions of hGH are deamination, oxidation, amino-terminal degradation and physical instability which produce multimeric (e.g., dimers) and more aggregated hGH forms. Deanimation is a post translational modification that results in the formation of desamidated hGH variants. Specifically, in deanimation, Asn149/Asp149 or Asn152/Asp152 becomes desamidated, and a less abundant desamido variant of glutamic acid residue 137 (hGHGlu 137) forms under exposure to alkalinity conditions. Commercial Norditropin® has been observed to include amounts of desamidated hGHAsp 152 upon storage. Desamidated forms of hGH have altered proteolytic cleavage sites, compared to non-desamidated hGH (Lewis et al. (1999), J. Biol. Chem., 274:7368-7378). More importantly, the physiological significance of altered proteolytic processing of hGH may result in the alteration of the physiological activity of the hGH in vivo.

Despite advances made in the manufacture of recombinant human growth hormone and growth hormone substitutes, a need remains in the medical arts for more efficient, commercially feasible higher yield methods for manufacturing with reduced amounts of potentially contaminating non-hGH species and/or that are less susceptible to formation of degradation products. Higher yield methods with fewer purification steps are needed for producing recombinant hGH more economically without loss of quality, preferably methods that do not require the use of environmentally harmful organic solvents. Reducing and/or eliminating formation of desamidated and other non-hGH forms during production reduces cost and time in manufacturing, and would greatly improve useful product yield. The present invention provides a solution to this need in the manufacture of recombinant hGH and in improved recombinant hGH products.

### SUMMARY

According to one aspect, the invention provides improved methods for manufacturing recombinant human growth hormone. In another aspect, improved preparations of human growth hormone as an active pharmaceutical ingredient (API) and in formulation are provided.

Method of Manufacture:

In some embodiments, the method for manufacturing an API of a recombinant human growth hormone comprises preparing a plasmid having a nucleic acid sequence encoding human growth hormone with a dipeptide Met-Asp appended to the N-terminus of the sequence; ligating said sequence of said plasmid into a suitable vector to provide a transformation vector having the dipeptide Met-Asp appended to the N-terminus of the sequence to provide a transformation vector; transforming E. coli with said vector to provide transformed E. coli; culturing said transformed E. coli in a nutrient media under appropriate pH to provide transformed E. coli starter cultures, growing said starting cultures to an A₆₀₀ (optical density) of 2 to 4 to provide mature E. coli cultures, inoculating a media within a fermentor with an amount of the mature E. coli culture; inducing the mature E. coli culture within the fermentor to provide induced mature E. coli that transcribe the hGH-dipeptide Met-Asp sequence within the fermentation media to reach an OD₆₀₀ of about 20; harvesting and concentrating the induced mature E. coli from the fermentor to provide an E. coli paste. The E. coli paste contains the transformed E. coli containing the MetAsp-hGH sequence, and may be stored away under appropriate conditions as a Master Cell Bank. By way of example, the frozen cell paste may be stored at −80° C. until time of use.

Alternatively, the E. coli paste containing the recombinant E. coli cells may be processed by culturing the recombinant E. coli cells and obtaining Met-Asp-hGH containing inclusion bodies therefrom. The inclusion bodies are isolated from the recombinant E. coli cells by lysing (in a Tris/salt lysis buffer) and homogenizing cultured E. coli recombinant cells in a cell lysis buffer and disrupting the recombinant E. coli to release the inclusion bodies; obtaining a concentrated fraction of released inclusion bodies and subjecting said inclusion bodies to a urea/salt wash, centrifugation step, and a detergent wash to provide detergent washed inclusion bodies in a detergent containing buffer, washing said detergent washed inclusion bodies to provide an essentially detergent free preparation of inclusion bodies, suspending said inclusion bodies in a Tris buffer, and preparing inclusion body fractions; solubilizing said inclusion body fraction in a high urea (8 mM) preparation to provide a solubilized inclusion body rich preparation, stirring and homogenizing said solubilized inclusion body rich preparation to release MetAsp hGH containing granules, refolding the MetAsp hGH released from the granules in the presence of a phosphate buffer by dialysis at an appropriate pH (about 8.0) and temperature (about 2-10° C.) to provide refolded MetAsp hGH; isolating a precipitate of the refolded Met Asp-hGH under appropriate pH (about pH 6.8-7.0); cleaving the MetAsp-hGH with the diaminopeptidase cathepsin C at an appropriate ratio thereto (ratio 1:3021 M) under appropriate conditions (37° C.) for an adequate amount of time (about 16-18 hours) to provide a cleaved hGH preparation (an hGH having a native human sequence without MetAsp); processing said cleaved hGH preparation over a chromatography column and collecting an ion exchanged hGH pooled fraction thereof; subjecting an adjusted preparation (made 5% with N-propanol, pH adjusted to 7.0) of the ion exchanged hGH pooled fraction to reverse phase chromatography to provide a RPHPLC pool; adjusting said RPHPLC pool to provide an adjusted RPHPLC pool (made 30% acetonitrile, pH adjusted to 6.8 to 8.0); desalting said adjusted RPHPLC pool; and lyophilizing said desalted RPHPLC pool to provide a purified recombinant hGH.

In some embodiments, the vector (plasmid) into which the MetAsp hGH sequence is inserted is pTrcHis2A/Kan.

In some embodiments of the method of manufacture where the Master Cell Bank (MCB) or Working Cell Bank of MetAsp hGH E. coli is to be used for production, an amount of the Master Cell Bank E. coli may be revived and cultured under suitable conditions in an appropriate cell culture media and processed to provide the recombinant MetAsp hGH as noted above. For example, and in some embodiments, this method may be further described as thawing an amount of MCB E. coli containing the pTrcHis2A/kan-MDNhGH clone prepared as descried above, inoculating a flask containing LBmedia/Kanamycin to provide a seed inoculum, growing the inoculum in a 37° C. environment while shaking at 250 rpm until the O.D.₆₀₀ nm reaches 2 to 4, inoculating a 2.5 L baffle flask containing TB Media/Kanamycin with 10 ml of inoculum/Liter of media, growing the cells until O.D.₆₀₀ nm reads 2.0-2.5, inducing the cells by adding 1 ml of 0.5M IPTG/Liter of TB media to provide induced recombinant E. coli cells, growing the induced cells for 3-4 hrs. to provide a concentrated preparation of recombinant cells and inclusion bodies, centrifuging the recombinant cells and media at 10,000-12,000×g for 15 min. and harvesting a centrifuged wet cell paste, and determining how many grams of wet cell paste is acquired. The wet cell paste can be stored at this stage at −80° C., or further processed to isolate MetAsp hGH containing inclusion bodies and stored at −80° C.

Recombinant hGH Preparations:

Preparations of the human hGH prepared as described herein have been found to provide a more stable and contaminant free preparation of human growth hormone. In some embodiments, the recombinant human growth hormone preparation comprises 2% or less of other than a recombinant human growth hormone, such as a desamidated hGH or undesirable multimeric or dimer form of hGH. In other embodiments, the recombinant human growth hormone preparation comprises 1% or less of other than a recombinant human growth hormone. In yet other embodiments, the recombinant human growth hormone preparation is essentially free of other than recombinant human growth hormone (essentially no contaminant or desamidated forms of hGH, as detectable using a reverse phase high pressure liquid chromatography (RPHPLC) analysis or ion exchange chromatography.

The following definitions are used in the description of the present invention:

As used in the description of the present invention, the term, “growth hormone” is used interchangeably with the term “somatotropin” (British: “somatotrophin”).

As used in the description of the present invention, the abbreviation “hGH” refers to human growth hormone and is an abbreviation for human growth hormone.

As used in the description of the present invention, the term “hydrophobic solvent” is defined as any chemical that would increase the hydrophobicity of a cell culture milieu.

Additional features and advantages are described herein, and will be apparent from, the following Detailed Description and the figures.

### BRIEF DESCRIPTION OF THE FIGURES

FIG. 1. SDS-PAGE Reduced Gel of Un-induced and induced analysis of MetAsp hGH expression.

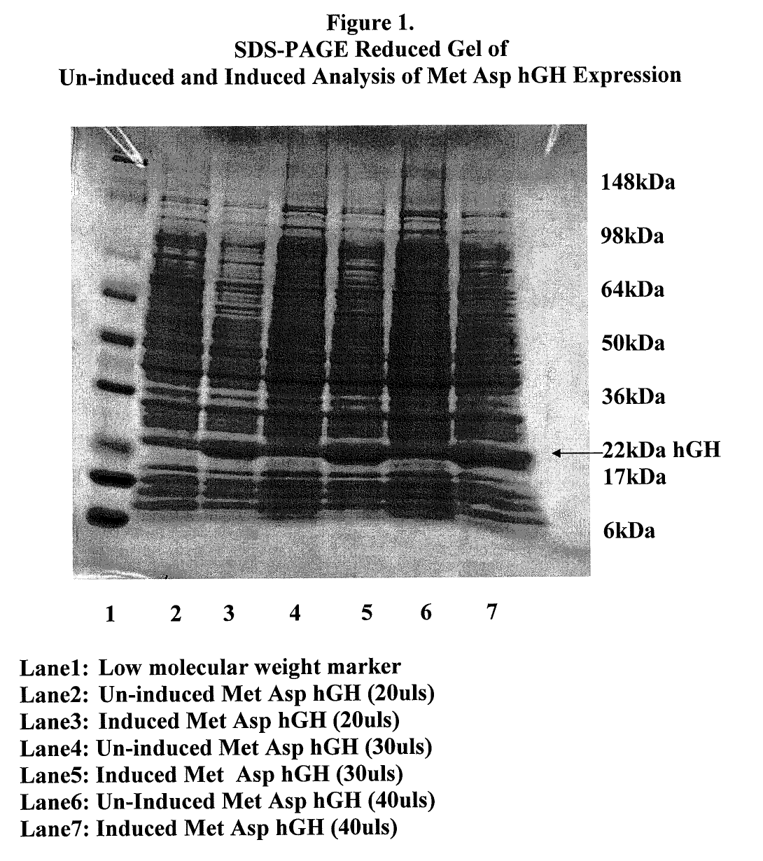

FIG. 2. SDS-PAGE Reduced Gel of Washed Inclusion Bodies of MetAsphGH. Gel displays the inventive hGH with a purity NLT (not less than) 70%.

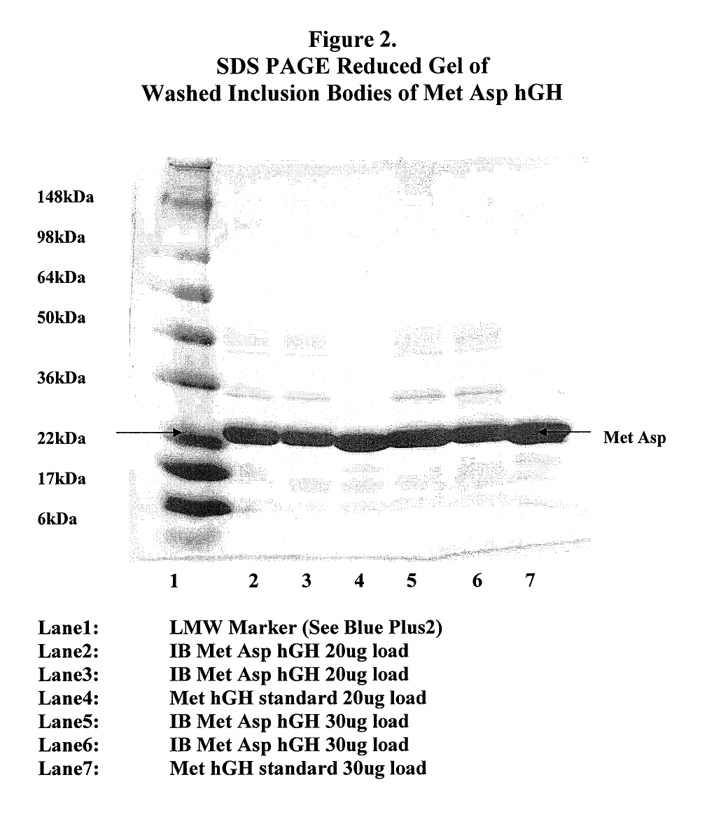

FIG. 3. Analytical Reverse phase high pressure liquid chromatography (RPHPLC) chromatogram demonstrating MetAsp-hGH being enzymatically digested by the diaminopeptidase Cathepsin C and native sequence hGH appearing with time.

FIG. 4. IEF Gel of the native sequence of human growth hormone. The gel shows that both Norditropin® and the presently described preparations of hGH contain only two isoforms. The lower pI band (5.2) represents the desamidated form of hGH and is readily more abundant in Norditropin® in contrast to inventive hGH (lane 8).

FIG. 5. Silver Stain Non-reduced SDS-PAGE Gel of Inventive hGH and Norditropin®.

FIG. 6A-6B. 6 A Analytical SEC of Norditropin® (b) and the prepared-hGH (c). The column was run in the presence of 30% acetonitrile. FIG. 6 B presents the enhancement of baseline. FIG. 6 A presents Norditropin® and the present hGH preparations. Both show a dimer which comprised less than 2%, and even less than 1%, of the preparation.

FIGS. 7A and 7B. Super-Q 5PW column from anion exchange chromatography results. Norditropin® shown to have an increased desamidated backside peak (5%) in contrast to the recombinant hGH of the present invention (lower levels of desamidated hGH (2%)). The leading backside peak appears to be where a desamidated form of the growth hormone might be expected to appear. FIG. 7B presents the enhancement of baseline. While not intended to be limited to any theory or mechanism of action, and in some embodiments of the preparations, compositions and formulations provided herein, the contaminant may at least in part comprise a desamidated form of hGH.

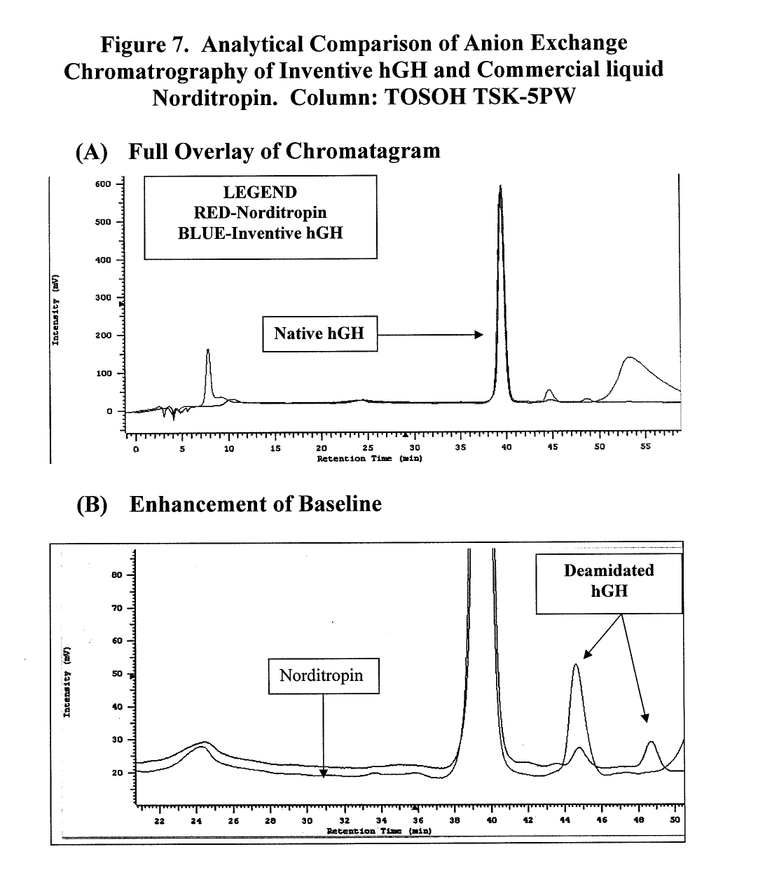

FIG. 8A-8B. FIG. 8A, an analytical reverse phase HPLC chromatogram of the prepared hGH vs. Norditrophin. FIG. 8B shows that the two samples are similar in purity. (Enhancement of baseline).

FIG. 9. SDS-PAGE gel results of the purification process of native growth hormone.

FIG. 10. Flow chart of Method of Manufacturing recombinant hGH.

FIG. 11. Comparison of the hGH prepared according to the present methods analytically to Norditropin® using Q-TOF Mass Spectrometry. The mass spectrometry results from the preparation protocol is presented. A small presence of MetAsp hGH was seen in the Norditropin® sample. Peptide mapping was done on both Norditropin® and the present preparation of hGH after they were digested with trypsin and show peptide maps.

FIG. 12. Peptide mapping done on both Norditropin® and the present preparation of hGH after trypsin digestion. The peptide maps show the peptides that were identified. The maps were essentially identical. MALDI TOF peptide mass fingerprint of inventive hGH. FIG. 12 discloses SEQ ID NOS 24, 15, 11, 6-7, 25, 20, 14, 18, 26, and 8-10, respectively, in order of appearance.

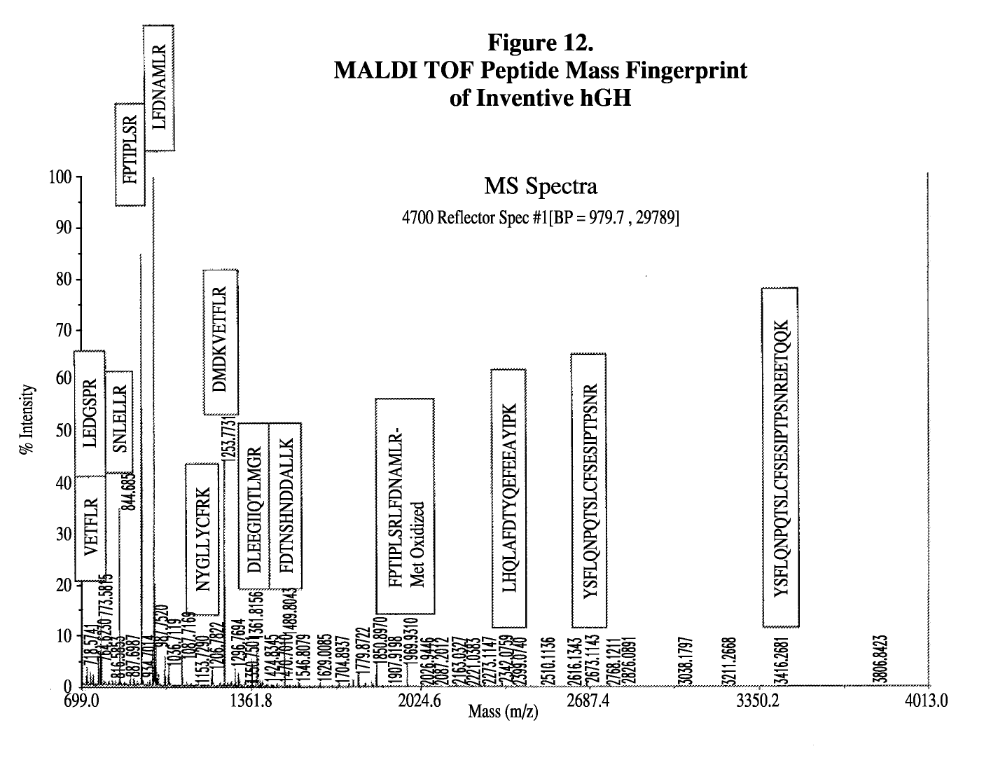

FIG. 13. Chromatogram of Q-Sepharose HP elution profile. Fractions were pooled by RP-HPLC analysis.

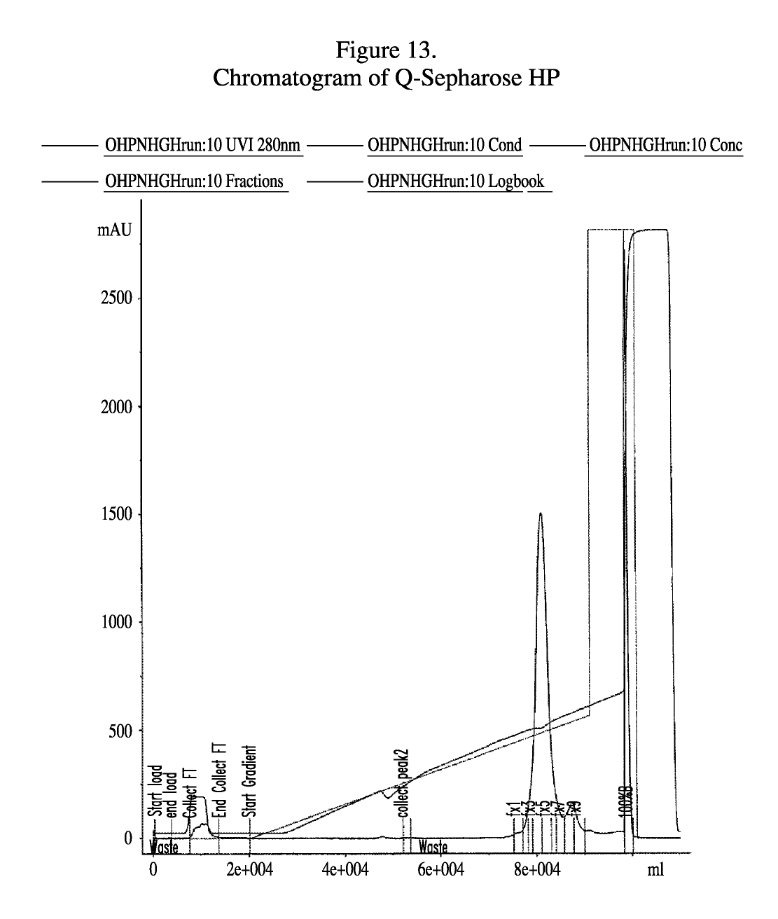

FIG. 14. Analytical RP-HPLC of Pooled Q-Sepharose HP main peak Elution.

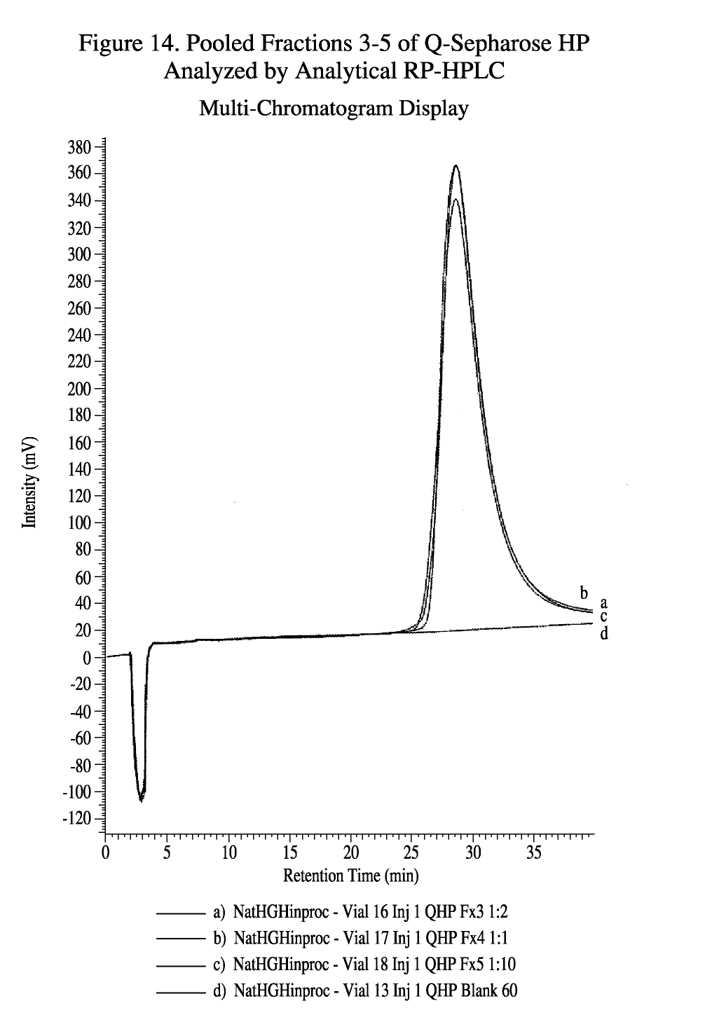

FIG. 15. Chromatogram of the RP-HPLC elution profile of native hGH.

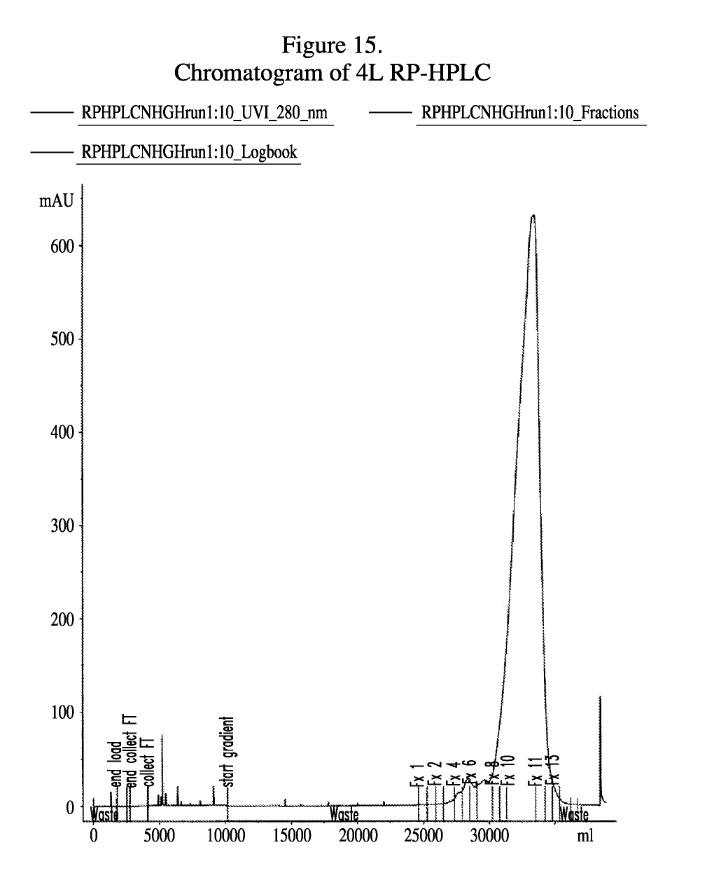

FIG. 16. Analytical RP-HPLC chromatogram of pooled RP Fractions prior to lyophilization.

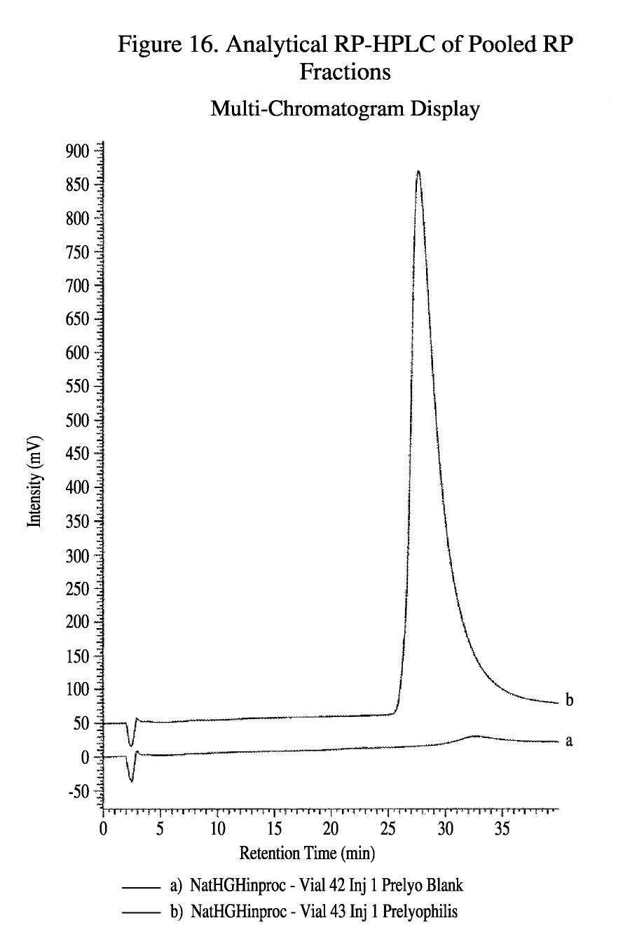

FIG. 17. Molecular construct, pTrcHis2aKan, having 5745 nucleotides, and a Multiple Cloning site at bases 411-464, a Trc promoter region at bases 190-382, a Lac operator (lacO) at bases 228-248, a ribosome binding site at bases 369-373 and a Kanamycin resistance gene at bases 1623-2437. FIG. 17 discloses “6×His” as SEQ ID NO:27.

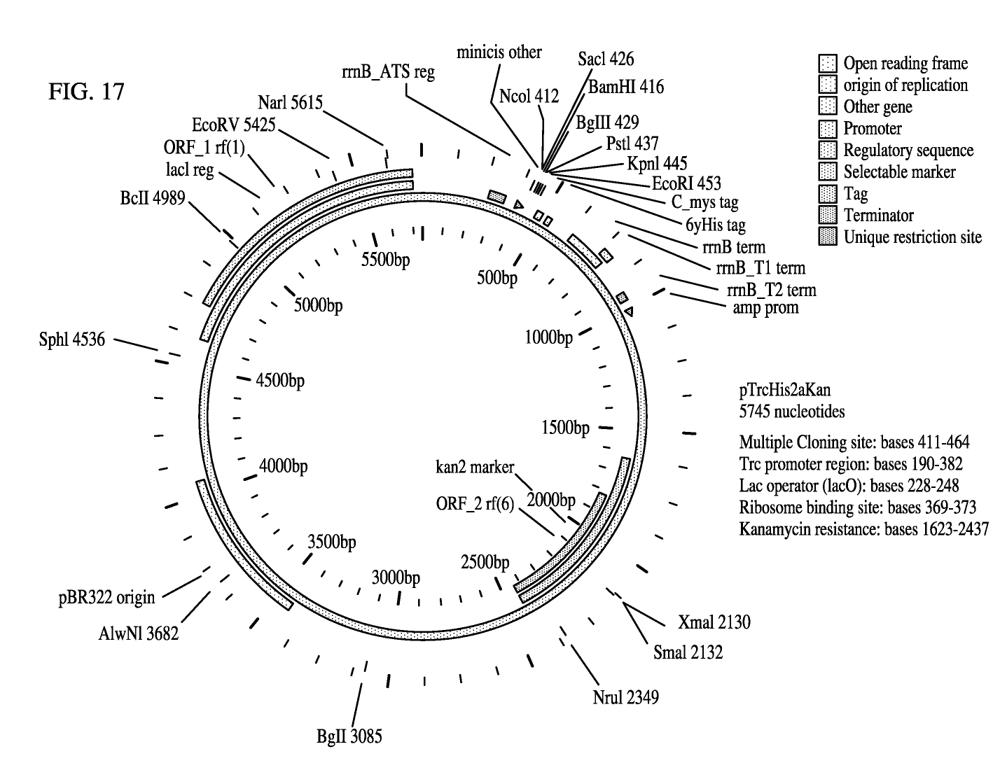

_ open reading

▪ Origin of replication

[symbol] a Other gene

[symbol] Promoter

[symbol] Regulatory sequence

[symbol] Selectable marker

□ Tag

▪ Terminator

□ Unique restriction site

FIG. 18. Molecular construct, pTrcHis2aKan/metasphGH. FIG. 18 discloses “6×His” as SEQ ID NO: 27.

### DETAILED DESCRIPTION

In one aspect, a pharmaceutical composition of recombinant human growth hormone (hGH) is provided comprising a recombinant native human growth hormone having less 2% or less of a non-native human growth hormone. In some embodiments, the non-native human growth hormone may include a desamidated hGH or other non-native hGH species (contaminant or degradative by-product).

In some embodiments, the pharmaceutical composition may comprise an active pharmaceutical ingredient API (non-formulated) or a formulated native recombinant hGH. In a formulated preparation of the recombinant hGH, the preparation will include a pharmaceutically acceptable carrier. These compositions can be administered by any means that achieve their intended purposes.

Amounts and regimens for the administration of a composition according to the present invention can be determined readily by those with ordinary skill in the art. By way of example, the compositions may be administered by a parenteral administration route, such as in a subcutaneous, intravenous, intramuscular, intraperitoneal, aerosol, or transdermal preparation. The dosage administered depends upon the age, health and weight of the recipient, type of previous or concurrent treatment, if any, frequency of the treatment and the nature of the effect desired.

Compositions within the scope of this invention include all compositions comprising at least one recombinant human growth hormone according to the present invention with reduced impurities and/or degradation product. Dosage amounts will vary with the condition being treated. While individual needs may vary, determination of optimal ranges of effective amounts of each component may be determined with only a reasonable amount of trial and error by the attending medical professional of skill in the art.

By way of example, typical dosages may comprise about 0.01 to about 0.1 mg/kg body weight per day. This is calculated to amount to about 16 mg/day, and may also be provided to a patient in need of hGH subcutaneously for 530 weeks.

In another aspect, the invention provides a method for treating human immunodeficiency virus diseases, for example, in the treatment of acquired immune deficiency syndrome (AIDS). When administered to AIDS patients, the hGH anti-HADDS therapy may be administered concomitantly with other AIDS therapies. Since supraphysiologic doses of hGH (>5 mg/day) have been safely administered to AIDS wasting patients continuously on a daily basis as s.c. injections for periods of two to four years (data on file, Serono Laboratories, Inc), in HADDS patients in whom the abnormal adipose tissue re-accumulates, re-treatment or maintenance therapies will be considered.

It should also be understood that, to be especially useful in some embodiments of the treatment, the treatment provided need not be absolute, provided that it is sufficient to provide at least some demonstrable measure of patient improvement and/or comfort as part of a clinically valuable treatment regimen. For example, a selected agent that provides a less effective treatment for a particular patient compared to another pharmaceutical agent may still be of value if the agent, when used in combination with other agents, enhances the overall level of protection, or if it is safer than competitive agents.

It is understood that the suitable dose of a composition according to the present invention will depend upon the age, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight.

The total dose required for each treatment may be administered in multiple doses or in a single dose. The compositions may be administered alone or in conjunction with other therapeutics directed to the disease or directed to other symptoms thereof.

In addition to the compounds of the invention, a pharmaceutical composition may contain suitable pharmaceutically acceptable carriers, such as excipients, carriers and/or auxiliaries which facilitate processing of the active compounds into preparations which can be used pharmaceutically.

Growth pre-hormone and growth hormone synthesized as described are purified by techniques well known in the art, including for example, gel filtration, ion exchange chromatography, affinity chromatography and differential solubility techniques.

The details of the present invention will be further described by the following examples. In these examples, digestions with restriction endonucleases were carried out under conditions optimized for each enzyme. Restriction endonucleases, their nomenclature and site specificity have been described in detail by Roberts, R., Crit. Rev. Biochem., 4, 123 (1976). Enzymes were obtained commercially (New England Biolabs, Cambridge, Mass.) and optimal conditions according to supplier's recommendations were employed unless noted otherwise. It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### Example 1

### Materials and Sequences Used in hGH Manufacturing Process

The present example is provided to describe the nucleic acid sequences employed to prepare the transformed E. coli clone (Master Cell Bank), hGH, and transformation vector used to transform E. coli.

pTreHis2A Kan-vector

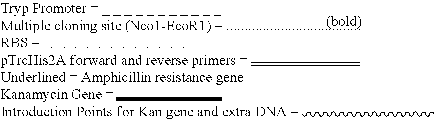

TABLE 1
Sequence of pTrcHis2AKan

GTTTGACAGCTTATCATCGACTGCACGGTGCACCAATGCTTCTGGCGTCA
GGCAGCCATCGGAAGCTGTGGTATGGCTGTGCAGGTCGTAAATCACTGCA
TAATTCGTGTCGCTCAAGGCGCACTCCCGTTCTGGATAATGTTTTTTGCG

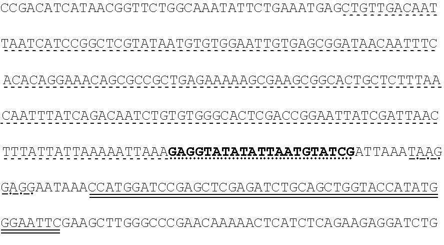

AATAGCGCCGTCGACCATCATCATCATCATCATTGAGTTTAAACGGTCTC

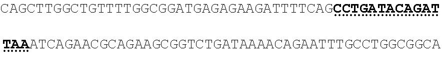

GTAGCGCGGTGGTCCCACCTGACCCCATGCCGAACTCAGAAGTGAAACGC
CGTAGCGCCGATGGTAGTGTGGGGTCTCCCCATGCGAGAGTAGGGAACTG
CCAGGCATCAAATAAAACGAAAGGCTCAGTCGAAAGACTGGGCCTTTCGT
TTTATCTGTTGTTTGTCGGTGAACGCTCTCCTGAGTAGGACAAATCCGCC
GGGAGCGGATTTGAACGTTGCGAAGCAACGGCCCGGAGGGTGGCGGGCAG
GACGCCCGCCATAAACTGCCAGGCATCAAATTAAGCAGAAGGCCATCCTG
ACGGATGGCCTTTTTGCGTTTCTACAAACTCTTTTTGTTTATTTTTCTAA
ATACATTCAAATATGTATCCGCTCATGAGACAATAACCCTGATAAATGCT
TCAATAATATTGAAAAAGGAAGAGTATGAGTATTCAACATTTCCGTGTCG
CCCTTATTCCCTTTTTTGCGGCATTTTGCCTTCCTGTTTTTGCTCACCCA
GAAACGCTGGTGAAAGTAAAAGATGCTGAAGATCAGTTGGGTGCACGAGT
GGGTTACATCGAACTGGATCTCAACAGCGGTAAGATCCTTGAGAGTTTTC
GCCCCGAAGAACGTTTTCCAATGATGAGCACTTTTAAAGTTCTGCTATGT
GGCGCGGTATTATCCCGTGTTGACGCCGGGCAAGAGCAACTCGGTCGCCG
CATACACTATTCTCAGAATGACTTGGTTGAGTCCTGAATCGCCCCATCAT
CCAGCCAGAAAGTGAGGGAGCCACGGTTGATGAGAGCTTTGTTGTAGGTG
GACCAGTTGGTGATTTTGAACTTTTGCTTTGCCACGGAACGGTCTGCGTT
GTCGGGAAGATGCGTGATCTGATCCTTCAACTCAGCAAAAGTTCGATTTA

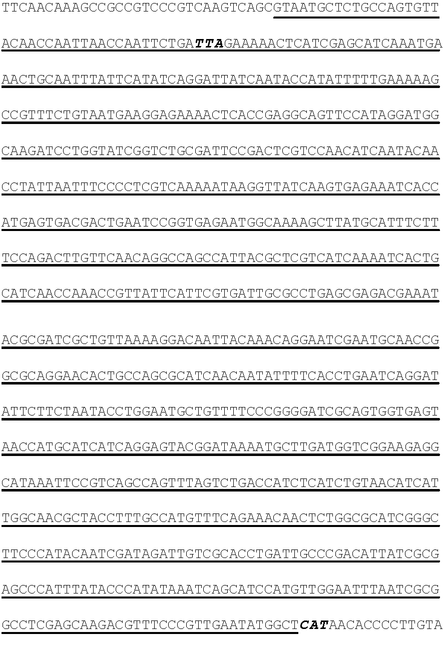

TTACTGTTTATGTAAGCAGACAGTTTTATTGTTCATGATGATATATTTTT
ATCTTGTGCAATGTAACATCAGAGATTTTGAGACACAACGTGGCTTTGTT
GAATAAATCGAACTTTTGCTGAGTTGAAGGATCAGATCACGCATCTTCCC
GACAACGCAGACCGTTCCGTGGCAAAGCAAAAGTTCAAAATCACCAACTG
GTCCACCTACAACAAAGCTCTCATCAACCGTGGCTCCCTCACTTTCTGGC

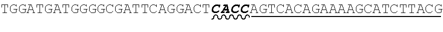

GATGGCATGACAGTAAGAGAATTATGCAGTGCTGCCATAACCATGAGTGA
TAACACTGCGGCCAACTTACTTCTGACAACGATCGGAGGACCGAAGGAGC
TAACCGCTTTTTTGCACAACATGGGGGATCATGTAACTCGCCTTGATCGT
TGGGAACCGGAGCTGAATGAAGCCATACCAAACGACGAGCGTGACACCAC
GATGCCTGTAGCAATGGCAACAACGTTGCGCAAACTATTAACTGGCGAAC
TACTTACTCTAGCTTCCCGGCAACAATTAATAGACTGGATGGAGGCGGAT
AAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAG
CACTGGGGCCAGATGGTAAGCCCTCCCGTATCGTAGTTATCTACACGACG
GGGAGTCAGGCAACTATGGATGAACGAAATAGACAGATCGCTGAGATAGG
TGCCTCACTGATTAAGCATTGGTAACTGTCAGACCAAGTTTACTCATATA
TACTTTAGATTGATTAAAACTTCATTTTTAATTTAAAAGGATCTAGGTTG
AAGATCCTTTTTGATAATCTCATGACCAAAATCCCTTAACGTGAGTTTTC
GTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTG
AGCTGATACCGCTCGCCGCAGCCGAACGACCGAGCGCAGCGAGTCAGTGA
GCGAGGAAGCGGAAGAGCGCCTGATGCGGTATTTTCTCCTTACGCATCTG
TGCGGTATTTCACACCGCATATGGTGCACTCTCAGTACAATCTGCTCTGA
TGCCGCATAGTTAAGCCAGTATACACTCCGCTATCGCTACGTGACTGGGT
CATGGCTGCGCCCCGACACCCGCCAACACCCGCTGACGCGCCCTGACGGG
CTTGTCTGCTCCCGGCATCCGCTTACAGACAAGCTGTGACCGTCTCCGGG
AGCTGCATGTGTCAGAGGTTTTCACCGTCATCACCGAAACGCGCGAGGCA
GCAGATCAATTCGCGCGCGAAGGCGAAGCGGCATGCATTTACGTTGACAC
CATCGAATGGTGCAAAACCTTTCGCGGTATGGCATGATAGCGCCCGGAAG
AGAGTCAATTCAGGGTGGTGAATGTGAAACCAGTAACGTTATACGATGTC
GCAGAGTATGCCGGTGTCTCTTATCAGACCGTTTCCCGCGTGGTGAACCA
GGCCAGCCACGTTTCTGCGAAAACGCGGGAAAAAGTGGAAGCGGCGATGG
CGGAGCTGAATTACATTCCCAACCGCGTGGCACAACAACTGGCGGGCAAA
CAGTCGTTGCTGATTGGCGTTGCCACCTCCAGTCTGGCCCTGCACGCGCC
GTCGCAAATTGTCGCGGCGATTAAATCTCGCGCCGATCAACTGGGTGCCA
GCGTGGTGGTGTCGATGGTAGAACGAAGCGGCGTCGAAGCCTGTAAAGCG
GCGGTGCACAATCTTCTCGCGCAACGCGTCAGTGGGCTGATCATTAACTA
TCCGCTGGATGACCAGGATGCCATTGCTGTGGAAGCTGCCTGCACTAATG
TTCCGGCGTTATTTCTTGATGTCTCTGACCAGACACCCATCAACAGTATT
ATTTTCTCCCATGAAGACGGTACGCGACTGGGCGTGGAGCATCTGGTCGC
ATTGGGTCACCAGCAAATCGCGCTGTTAGCGGGCCCATTAAGTTCTGTCT
CGGCGCGTCTGCGTCTGGCTGGCTGGCATAAATATCTCACTCGCAATCAA
ATTCAGCCGATAGCGGAACGGGAAGGCGACTGGAGTGCCATGTCCGGTTT
TCAACAAACCATGCAAATGCTGAATGAGGGCATCGTTCCCACTGCGATGC
TGGTTGCCAACGATCAGATGGCGCTGGGCGCAATGCGCGCCATTACCGAG
TCCGGGCTGCGCGTTGGTGCGGATATCTCGGTAGTGGGATACGACGATAC
CGAAGACAGCTCATGTTATATCCCGCCGTCAACCACCATCAAACAGGATT
TTCGCCTGCTGGGGCAAACCAGCGTGGACCGCTTGCTGCAACTCTCTCAG
GGCCAGGCGGTGAAGGGCAATCAGCTGTTGCCCGTCTCACTGGTGAAAAG
AAAAACCACCCTGGCGCCCAATACGCAAACCGCCTCTCCCCGCGCGTTGG
CCGATTCATTAATGCAGCTGGCACGACAGGTTTCCCGACTGGAAAGCGGG
CAGTGAGCGCAACGCAATTAATGTGAGTTAGCGCGAATTGATCTG
(SEQ ID NO: 1)

TABLE 2
Gene for Native Growth Hormone ligated between NcoI and EcoRI

TCGACCGGAATTATCGATTAACTTTATTATTAAAAATTAAAGAGGTATATATTAATGTATCGATTAAATAAGGAGG
AATAAACC
ATGGACTT CCCAACTATC CCACTGTCTC GTCTGTTCGA TAACGCTATG CTTCGTGCTC ATCGTCTTCA
TCAGCTGGCC TTTGACACCT ACCAGGAGTT TGAAGAAGCC TATATCCCAA AGGAACAGAA GTATTCATTC
CTGCAGAACC CGCAGACCTC CCTCTGTTTC TCAGAGTCTA TTCCGACCC GTCCAACCGT GAGGAAACAC
AACAGAAATC CAACCTGGAG CTGCTCCGCA TCTCCCTGCT GCTCATCCAG TCGTGGCTGG AGCCGGTGCA
GTTCCTCCGT AGTGTCTTCG CCAACAGCCT GGTGTACGGC GCCTCTGACA GCAACGTCTA TGACCTCCTG
AAGGACCTGG AGGAAGGCAT CCAAACCCTG ATGGGTCGTC TGGAAGATGG CAGCCCGCGT ACTGGTCAGA
TCTTCAAGCA GACCTACAGC AAGTTCGACA CAAACTCACA CAACGATGAC GCACTGCTCA AGAACTACGG
TCTGCTCTAC TGCTTCCGTA AGGACATGGA CAAGGTCGAG ACATTCCTGC GCATCGTGCA GTGCCGCTCT
GTGGAGGGCA GCTGTGGCTT CTAG
GAATTCGAAGCTTGGGCCCGAACAAAAACTCATCTCAGAAGAGGATCTGAATAGCGCCGTCGACCATCATCATCAT
CATCATTGAGTTTAAACGGTCTCCAGCTTGGCTGTTTTGGCGGA (SEQ ID NO: 2)

TABLE 3
Gene Sequence (Native Human Growth
Hormone)-(Nucleic acid sequence for
human growth hormone)

ATGGACTT CCCAACTATC CCACTGTCTC GTCTGTTCGA
TAACGCTATG CTTCGTGCTC ATCGTCTTCA TCAGCTGGCC
TTTGACACCT ACCAGGAGTT TGAAGAAGCC TATATCCCAA
AGGAACAGAA GTATTCATTC CTGCAGAACC CGCAGACCTC
CCTCTGTTTC TCAGAGTCTA TTCCGACACC GTCCAACCGT
GAGGAAACAC AACAGAAATC CAACCTGGAG CTGCTCCGCA
TCTCCCTGCT GCTCATCCAG TCGTGGCTGG AGCCGGTGCA
GTTCCTCCGT AGTGTCTTCG CCAACAGCCT GGTGTACGGC
GCCTCTGACA GCAACGTCTA TGACCTCCTG AAGGACCTGG
AGGAAGGCAT CCAAACCCTG ATGGGTCGTC TGGAAGATGG
CAGCCCGCGT ACTGGTCAGA TCTTCAAGCA GACCTACAGC
AAGTTCGACA CAAACTCACA CAACGATGAC GCACTGCTCA
AGAACTACGG TCTGCTCTAC TGCTTCCGTA AGGACATGGA
CAAGGTCGAG ACATTCCTGC GCATCGTGCA GTGCCGCTCT
GTGGAGGGCA GCTGTGGCTT CTAG (SEQ ID NO: 3)

TABLE 4
Protein sequence for hGH
(Amino acid sequence for human growth hormone)

MDFPTIPLSRLFDNAMLRAHRLHQLAFDTYQEFEEAYIPKEQKYSFLQ
NPQTSLCFSESIPTPSNREETQQKSNLELLRISLLLIQSWLEPVQFLR
SVFANSLVYGASDSNVYDLLKDLEEGIQTLMGRLEDGSPRTGQIFKQT
YSKFDTNSHNDDALLKNYGLLYCFRKDMDKVETFLRIVQCRSVEGSCG
F-(SEQ ID NO: 4)

TABLE 5
pTrcHis2A/kan-MDNhGH plasmid vector (Nucleic Acid sequence for
pTrcHis2A/kan-MDNhGH plasmid)

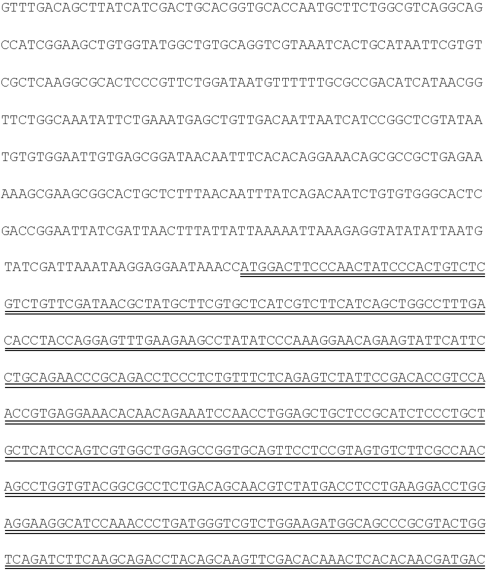

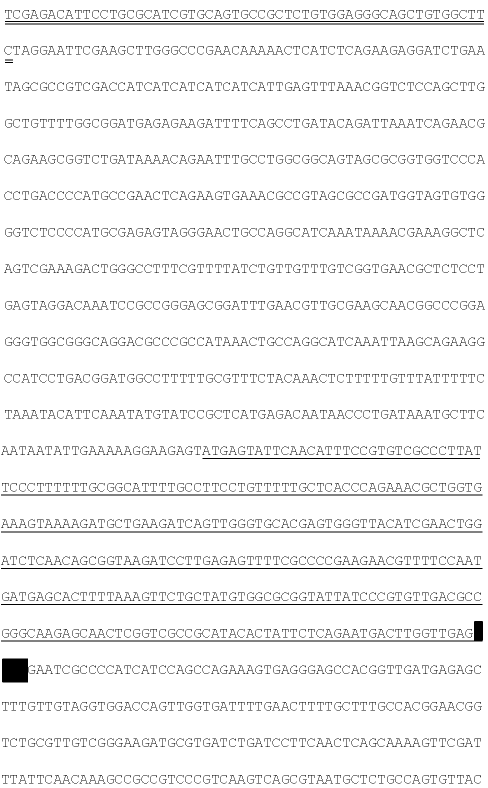

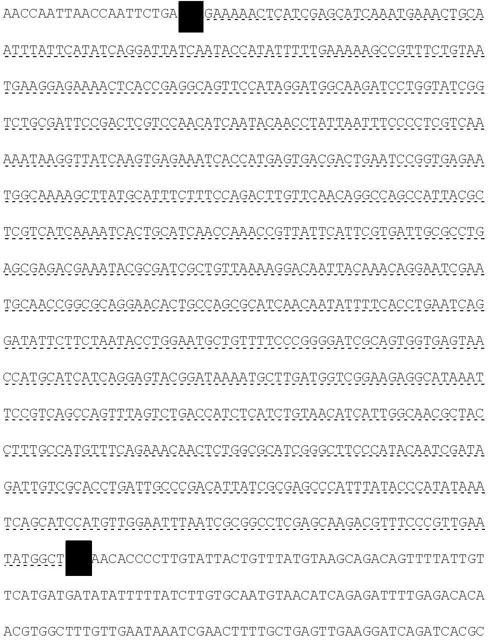

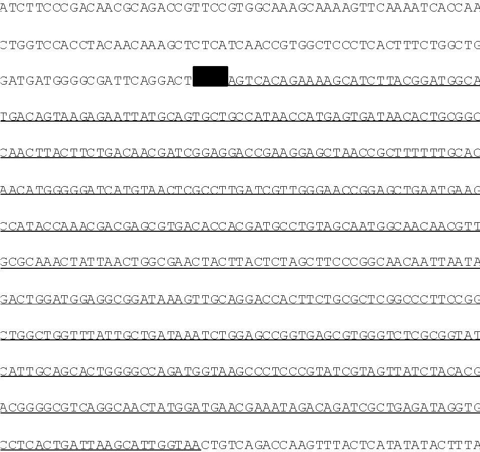

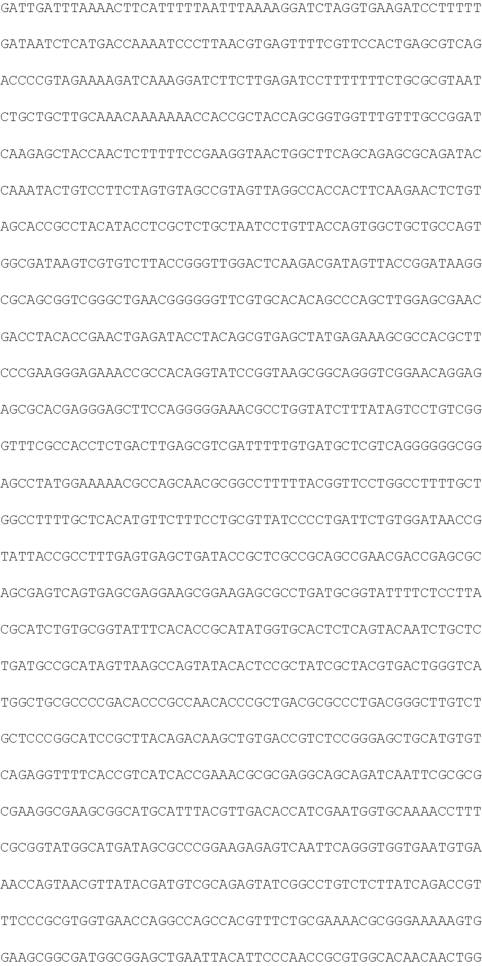

CGGGCAAACAGTCGTTGCTGATTGGCGTTGCCACCTCCAGTCTGGCCCTGCACGC
GCCGTCGCAAATTGTCGCGGCGATTAAATCTCGCGCCGATCAACTGGGTGCCAGC
GTGGTGGTGTCGATGGTAGAACGAAGCGGCGTCGAAGCCTGTAAAGCGGCGGTGC
ACAATCTTCTCGCGCAACGCGTCAGTGGGCTGATCATTAACTATCCGCTGGATGA
CCAGGATGCCATTGCTGTGGAAGCTGCCTGCACTAATGTTCCGGCGTTATTTCTT
GATGTCTCTGACCAGACACCCATCAACAGTATTATTTTCTCCCATGAAGACGGTA
CGCGACTGGGCGTGGAGCATCTGGTCGCATTGGGTCACCAGCAAATCGCGCTGTT
AGCGGGCCCATTAAGTTCTGTCTCGGCGCGTCTGCGTCTGGCTGGCTGGCATAAA
TATCTCACTCGCAATCAAATTCAGCCGATAGCGGAACGGGAAGGCGACTGGAGTG
CCATGTCCGGTTTTCAACAAACCATGCAAATGCTGAATGAGGGCATCGTTCCCAC
TGCGATGCTGGTTGCCAACGATCAGATGGCGCTGGGCGCAATGCGCGCCATTACC
GAGTCCGGGCTGCGCGTTGGTGCGGATATCTCGGTAGTGGGATACGACGATACCG
AAGACAGCTCATGTTATATCCCGCCGTCAACCACCATCAAACAGGATTTTCGCCT
GCTGGGGCAAACCAGCGTGGACCGCTTGCTGCAACTCTCTCAGGGCCAGGCGGTG
AAGGGCAATCAGCTGTTGCCCGTCTCACTGGTGAAAAGAAAAACCACCCTGGCGC
CCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAG
TTAGCGCGAATTGATCTG (SEQ ID NO: 5)
6288 bp

### Example 2

### Purification and Production of Human Growth Hormone

The present example is provided to demonstrate the utility of the invention for providing a method of manufacture for an API of human growth hormone that has a reduced percent of product contaminant as measured by RP HPLC peak. The present example demonstrates the production of a recombinant human growth hormone that demonstrates a single spike in HPLC analysis, and is demonstrated to have fewer amounts of non-hGH related substances in contrast to commercial Norditropin®. Norditropin® is demonstrated to produce an RP HPLC having increased levels of non-hGH related substances. Such evidences the presence of a contaminant in commercial grade preparations that is not present in preparations of recombinant hGH produced according to the present invention.

Norditropin® was acquired from a commercial source. The MetAsp hGH was isolated from the transformed recombinant E. coli as described herein, and the N-terminal methionine removed by cleavage of an N-terminal dipeptide using cathepsin-C (dipeptidyl amino peptidase). This preparation was used to compare the human growth hormone preparation produced according to the present methods.

Preparation of the Expression Vector

Step 1—Construction of the pTrcHis2Kan Expression Vector

An optimized E. coli synthetic gene sequence for MetAsp hGH having a dipeptide MetAsp appended to the N-terminus was synthesized by DNA 2.0 (Menlo Park) according to specifications provided to the vendor. The gene for MetAsp hGH (See Table 3) was provided in the form of a circular plasmid. The full length gene sequence included Nc0I and EcoRI sites for ligation into the pTrcHis2A/Kan expression vector.

A Kan R expression vector was prepared by modifying a pTrcHis2A vector commercially available (Invitrogen, Carlsbad, Calif.) that had been modified to remove the ampicillin resistance gene and to substitute therein a kanamycin antibiotic resistance gene (performed by ATG Laboratories). This provided a pTrcHis2/KAN vector.

The MetAsp hGH sequence was ligated into the recipient vector to provide the expression vector pTrcHis2A/Kan-MetAsphGH.

Step 2—Preparation of Transformed E. coli

The expression vector was used to transformed into either BL21 or Top-10 E coli cells. In some embodiments, BL21 E. coli cells are preferred. Clones were selected by positive verification by DNA sequence analysis. Glycerol stocks of positive clones were stored at −80° C.

Preparation of Recombinant MetAsp hGH Master Cell Bank (MCB):

Starter inoculums of transformed E. coli were prepared and grown in a 0.25 L shake flask containing TB/Kan Medium (plant source) at 37° C. The starter cultures were grown at 37° C. at 250 PRM to an A₆₀₀ of 2-3. The 0.25 L cell culture was harvested by sterile filtration and re-suspended in 100 mL of fresh medium containing a cryprotectant (15% glycerol). Cell viability was determined and then aliquoted into 1 ml aliquotes in labeled cryogenic ampoules. The Master Cell Bank was stored at −80° C. Qualification of the Master Cell Bank was performed by Beckman Coulter Genomics. The Master Cell Bank is used to inoculate all seed cultures until a Working Cell Bank is established. A 1 L seed culture was used to inoculate a 100 L tank of sterile Terrific Broth in a New Brunswick Bio6000 Fermentor. Cells were fermented to an OD₆₀₀ 8.0-10.0 over a 5-6 hour period. The fermentation culture was induced with IPTG (to induce the transcription of met-asp hGH), and allowed to grow for an additional 3 hours. The final OD₆₀₀ of the fermentation broth was about 20. Cells were harvested by continuous flow centrifugation and the E. coli. cell pellet (2.2 kg wet cell paste) was stored at −80° C. Each 1 liter of fermentation broth produced approximately 22 grams of wet cell pellet containing about 660 mg of MetAsp hGH. A second fermentation run produced similar results as described above. This provided ample frozen E. coli expressed with MetAsp hGH to be used in the production of material to support both animal and human clinical trial material.

Purification of Human Growth Hormone

Approximately 2.2 kilograms of cell pellet was removed from the freezer and re-suspended into chilled 50 mM Tris-HCl, 1 mM EDTA, 0.2M NaCl at pH 8.0 (Cell lysis buffer). The volume of cell lysis buffer per 1 gram of cell pellet was 5 milliliters (total of 11 L) per gram of wet cell paste. The cell pellet was thawed in the lysis buffer and stirred with “lightning” mixer for NLT 30 minutes. A tissuemizer was used to homogenize the material until the slurry was uniform (NLT 5 minutes). At the end of the homogenization process, the cells were disrupted twice by pressure at 1000-1200 Bar using a Niro Press. The disrupted cells were centrifuged at 12,000g for not less than (NLT) 20 minutes at 2-10° C. The pellet containing inclusion bodies was collected and re-suspended and homogenized for NLT 4 minutes into 11 L of 2M Urea/1M NaCl in 50 mM Tris-HCl at pH 8. The urea/salt wash was centrifuged at 12,000g for 20 minutes at 2-10° C. The supernatant was discarded and the pellet re-suspended (homogenized) in 11 L of 50 mM Tris pH 8 with 0.25% Triton X100 (Triton Wash Buffer). The pellet was centrifuged at 12,000g for 20 minutes at 2-10° C. The supernatant was discarded and the cell pellet re-suspended (homogenized) into 11 L of USP purified water to remove the majority of the detergent. The water wash was centrifuged at 12,000 g for 30 minutes. The supernatant was discarded and the inclusion body pellet was re-suspended into 50 mM Tris-HCl pH 8 and homogenized until a homogenous slurry was achieved. The total wet inclusion body (IB) weight was 656.4 g. The final slurry volume was 1.2 L in 50 mM Tris-HCl pH 8.0. The slurry was divided into three equal portions (0.4 L) and stored at −80° C.

Inclusion bodies (approximately 0.8 L) were thawed at 4° C. at room temperature NLT 8 hours and were solubilized in 39 L of de-ionized 8 M urea in 50 mM Tris-HCl pH 8.0, 5 mM DTT, and 10 mM cysteine at a level of about 200-400 ug/mL of protein and stirred at room temperature for 30 minutes. The solubilized granules were poured into a series of 12,000-14,000 MWCO dialysis bags. The solubilized MetAsp-hGH granules were refolded by three dialysis exchanges of 1:4.625 for NLT 12 hours into 20 mM Phosphate buffer at pH 8.0 resulting in a final urea concentration of about 80 mM at 2-10° C. The final volume of refolded MetAsp hGH was about 47.5 L. The refolded MetAsp hGH was then concentrated to a final volume of 3.5 L (15.5 mg/ml) using a 10 kD Millipore Prepscale UF cartridge.

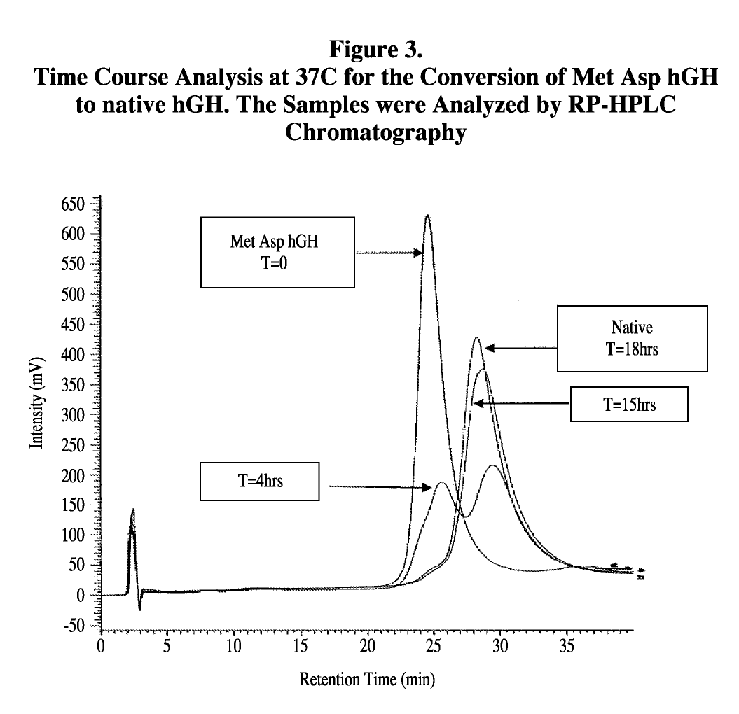

Refolded MetAsp-hGH was pH adjusted to pH 6.8 by titrating with phosphoric acid, and a copious precipitate appeared. This was removed by centrifugation at 12,000 g for 30 minutes at 2-10° C. The concentration of MetAsp hGH by UV₂₈₀ analysis was determined to be about 52 g. The enzymatic cleavage of Met-asp-hGH to Native hGH was performed using the diamminopeptidase Cathepsin C to remove the N-terminal MetAsp. The enzymatic reaction mixture is 20 mM Phosphate Buffer, 2 mM NaCl, 1 ug/mL Leupeptin, 5 mM 2-Mercaptoethanol and 140 Units of Cathepsin C/1 g of MetAsp hGH. The enzymatic reaction is stirred slowly at 37° C. and monitored by analytical RP-HPLC for depreciation of the MetAsp hGH peak and the enhancement of Native hGH peak. FIG. 3 shows RP-HPLC chromatography of MetAsp-hGH being removed and native sequence hGH appearing over time. The reaction is near completion at approximately 16-18 hours. The cleaved hGH material was pH adjusted with 0.5 M NaOH to pH 8.3 and filtered prior to column chromatography.

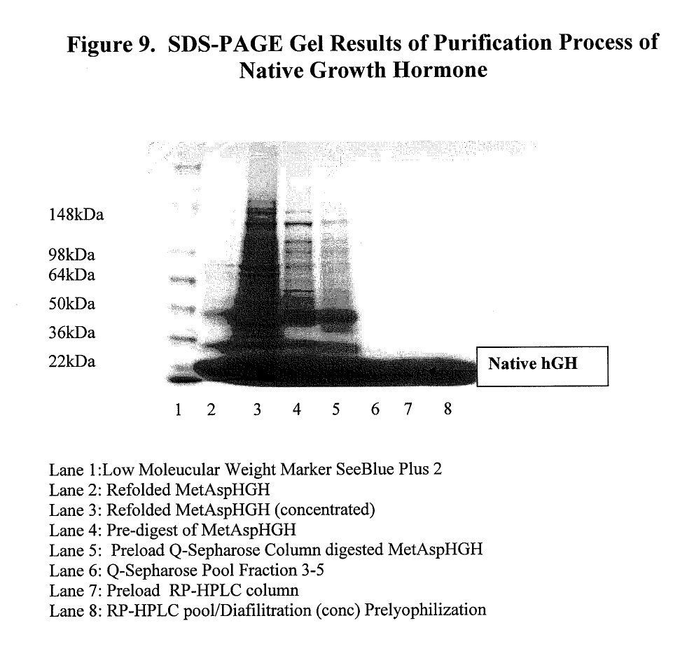

Cleaved material was chromatographed on a Q-Sepharose HP resin (8.8 L) in 50 mM Tris-HCl pH 8.3 and eluted from the column with a 12 column volume gradient of 0-0.3 M NaCl. Column fractions were monitored by RP-HPLC and those containing native hGH at least 98% by purity were pooled. The ion exchange pool was made 5% with N-propanol and pH adjusted to pH 7.0 and further purified on a C4 reverse phase column equilibrated in a 50% mixture of Buffer A (50 mM Tris, 5% N-Propanol, pH 7.0) & B (50 mM Tris 47.5% N-Propanol, pH 7.0). A 12 column volume gradient from 50%-90B % was performed. The fractions containing native hGH were pooled by analytical RP-HPLC methods. The RP-HPLC pool was adjusted to 30% Acetonitrile then pH adjusted from 6.8 to 8.0. The adjusted RP-HPLC pool was desalted by UF filtration into water adjusted to pH 8.0 with ammonium hydroxide. The desalted pool was then bulk lyophilized. Table 6 shows step yields during the purification of hGH along with final purity as determined by RPHPLC. FIG. 9 shows polyacrylamide gel electrophoresis of the pooled fractions for each step of purification.

Conventional methods employ the use of acetonitrile to minimize multimeric quantities of hGH at the ion exchange step. However, the present methods may be employed with an ion exchange step without the presence of acetonitrile without the high problematic incidence of multimeric forms in production.

The purification provided here for native hGH can be readily scaled to produce larger quantities of hGH by increasing the size of the bioprocess platform. It has been run a sufficient number of times to show considerable robustness and repeatability.

While conventional methods employ the use of acetonitrile to minimize multimeric forms of hGH at the ion exchange step, this procedure does the step in the absence of acetonitrile.

TABLE 6
Table 6 - Percent Step Yield Loss and Percent Purity
During the Manufacturing Process of recombinant hGH

| | Growth | | Overall | |
|---|---|---|---|---|
| | Hormone | Step Yield | Yield | |
| Step Process | (g) | loss (%) | (%) | % Purity |
| Refolded Met Asp hGH | 55 | — | 100 | 70 |
| Enzymatic digest | 52 | 9 | 91 | 70 |
| Anion Exchange Pool | 29 | 44 | 53 | 95 |
| RPHPLC Pool | 25 | 14 | 45 | NLT 98.5 |
| Buffer Exchange | 24 | 5 | 44 | NLT 98.5 |
| Bulk Lyophilization | 22 | 8 | 40 | NLT 98.5 |

### Example 2

### Analytical Comparison with Norditropin®

The hGH prepared according to the present methods was compared analytically to Norditropin® using the analytical methods of IEX, RPHPLC, SDS-PAGE, IEF, Mass Spec., and peptide mapping. FIG. 3A shows an analytical reverse phase RP HPLC chromatogram of hGH (broken (hatched) line) versus Norditropin® (solid line). FIG. 3B is the same chromatogram of hGH (broken (hatched) line) versus Norditropin® (solid line). The Y axis has been expanded to more clearly indicate contaminants. From FIG. 3B it is clear that Norditropin® had a leading edge contaminant that was not present in the present preparations of hGH. Integration of the RPHPLC of the two samples shows that the present hGH has a 99% main peak while Norditropin® has only a 98% peak.

### Example 3

### Ion Exchange Comparison with Norditropin

The hGH prepared according to the present methods was compared analytically to Norditropin® using an ion exchange chromatography analysis.

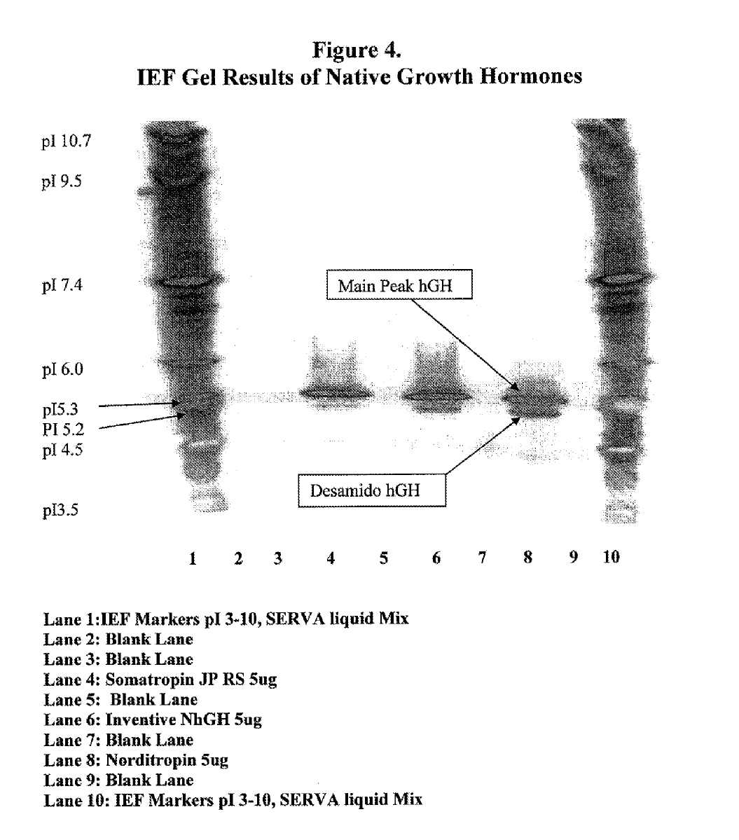

Ion exchange chromatography on a Super Q 5PW column from Tosoh Haas was done (FIGS. 4A and 4B). Ion exchange chromatography indicated that Norditropin® (solid line) had a leading peak that was not present in the present hGH preparations (broken (hatched) line). This peak appears to be where one would expect a desamidated form. The desamidated forms would typically be removed by IEX chromatography. There are multiple desamidated forms. These desamidated forms have been shown to have biological activity equivalent to the native form, but to have greater antigenicity.

### Example 4

### Analytical Size Exclusion Chromatography (SEC) Analysis

The recombinant hGH prepared according to the present methods was compared analytically to Norditropin® using analytical size exclusion chromatography (SEC).

Analytical size exclusion chromatography was done on a Tosoh Haas TSK-2000 column run both in the presence and in the absence of acetonitrile.

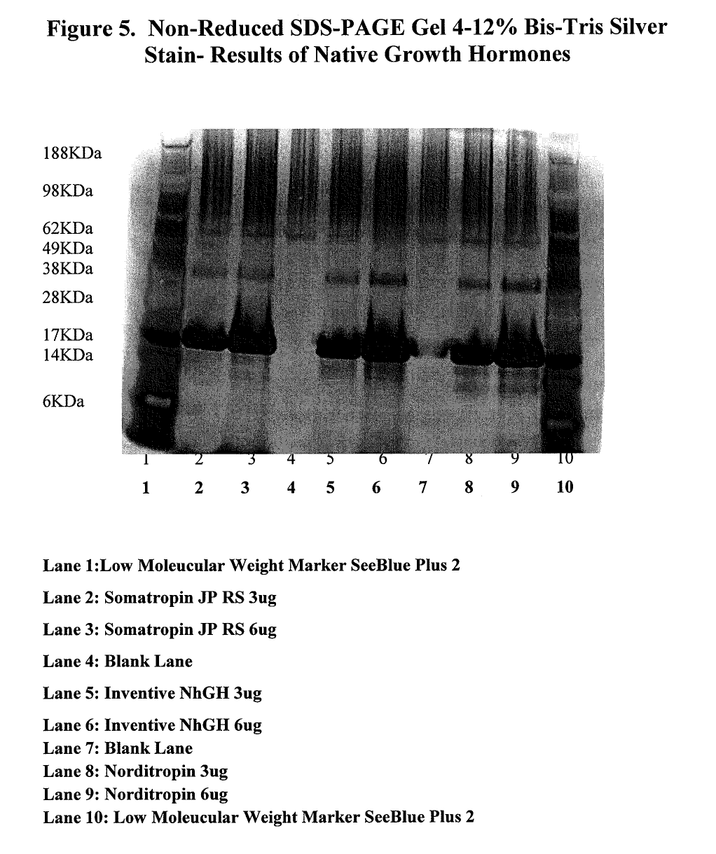

Acetonitrile has been shown to disrupt reversible hydrophobic aggregates. FIGS. 5A and 5B present the SEC of Norditropin® (solid line) and the prepared hGH (broken (hatched) line). The column was run in the absence of 30% acetonitrile.

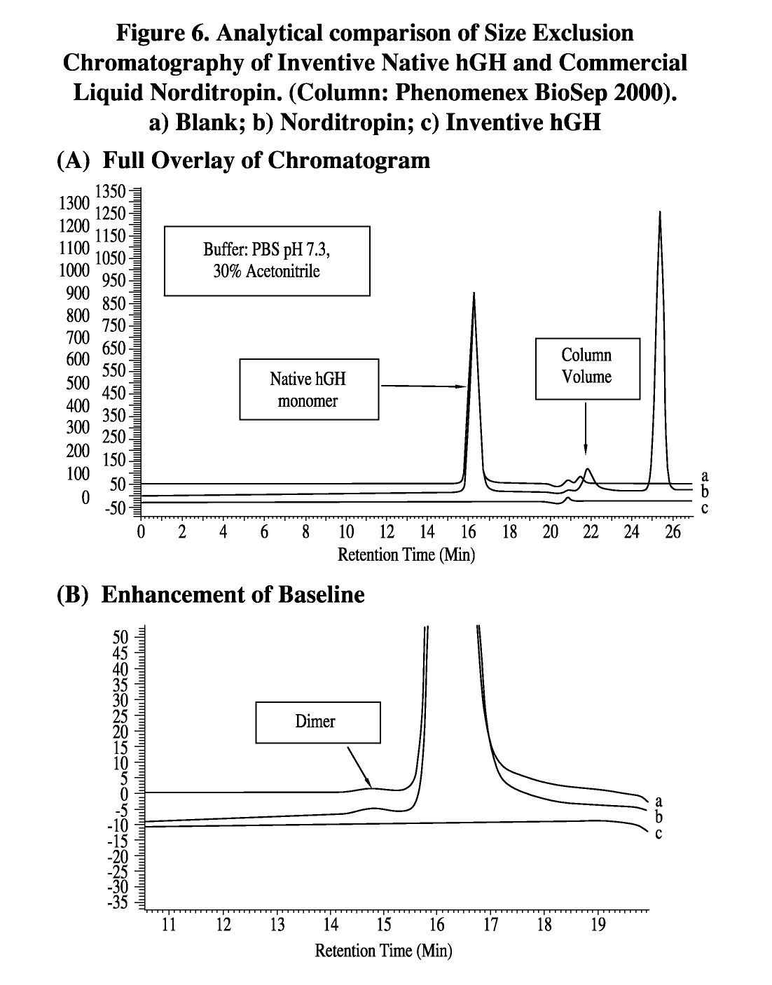

Norditropin® and the present hGH preparations both show a dimer and a higher molecular weight multimer. When the SEC was run in the presence of 30% acetonitrile (FIGS. 6A and 6B), the multimer was absent. The dimer was still present in both samples. Norditropin® had a low molecular weight that eluted at the column volume. This would indicate that a salt or other small molecule was present. The salt is primarily due to the particular formulation.

### Example 5

### Isoelectric Focusing Gels

The hGH prepared according to the present methods was compared analytically to Norditropin® using isoelectric focusing gels (IEF).

Both samples showed two bands at the predicted isoelectric point for human growth hormone (pI 5.3) and des-amido growth hormone (pI 5.2).

### Example 6

### Maldi-TOF Mass Spectrometry

The hGH prepared according to the present methods was compared analytically to Norditropin® using Maldi-TOF Mass Spectrometry.

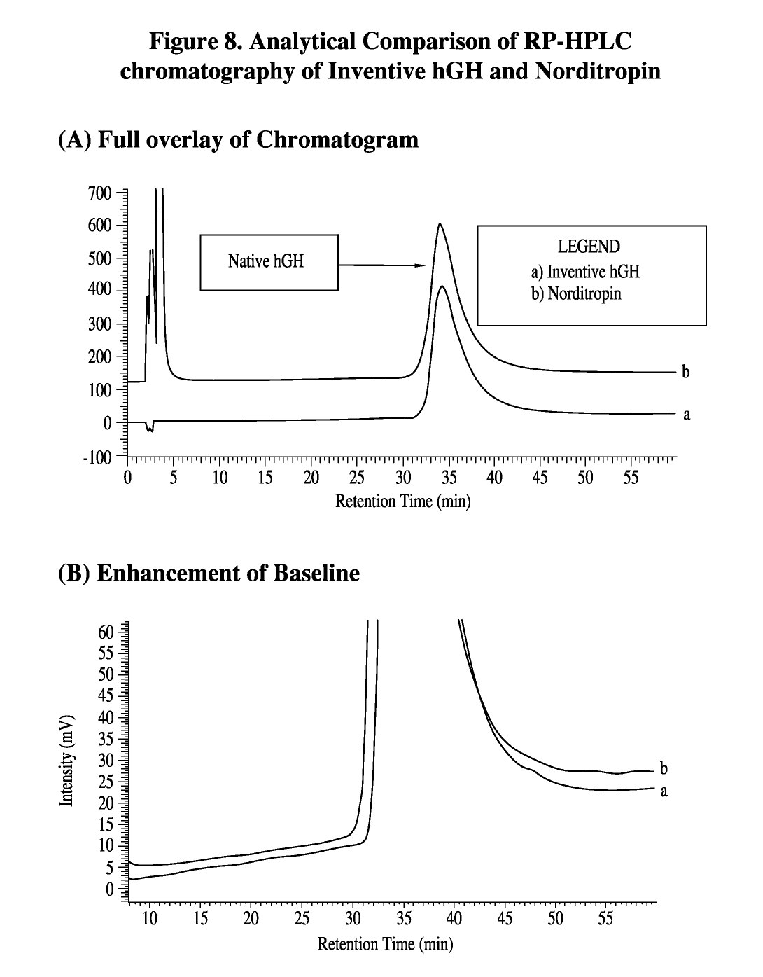

FIG. 8 shows the mass spectroscopy results from the preparation protocol described here. FIG. 9 presents the Maldi-TOF mass spectrometric results using the Norditropin®. Both of the samples were primarily native sequence hGH with a small amount of uncleaved MetAsp-hGH in which the met had oxidized to form a met-sulfoxide.

### Example 7

### Peptide Mapping

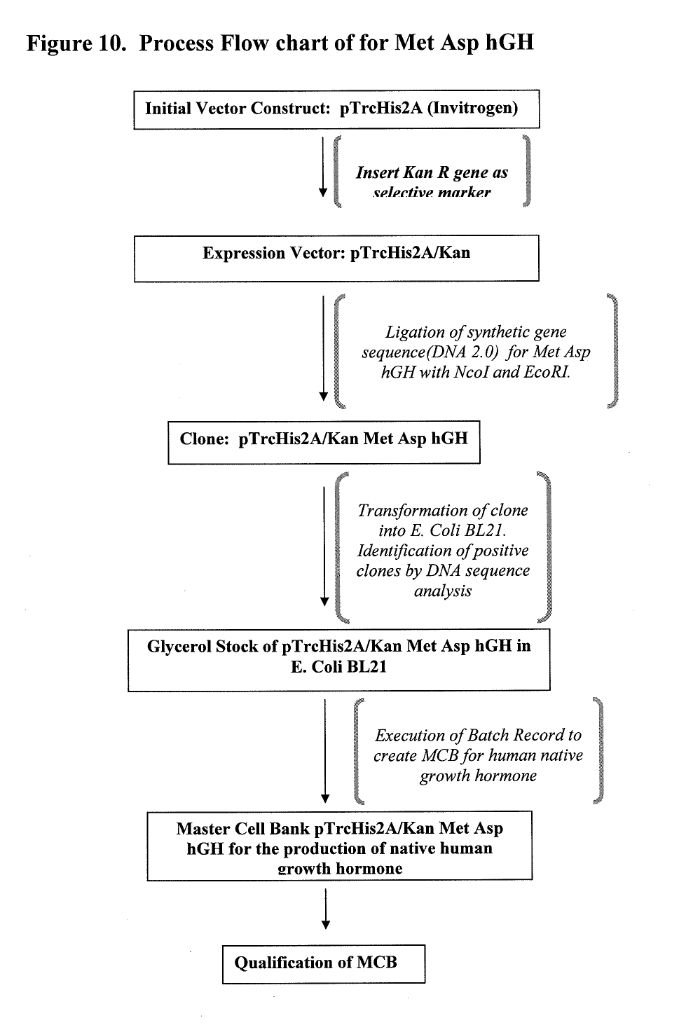

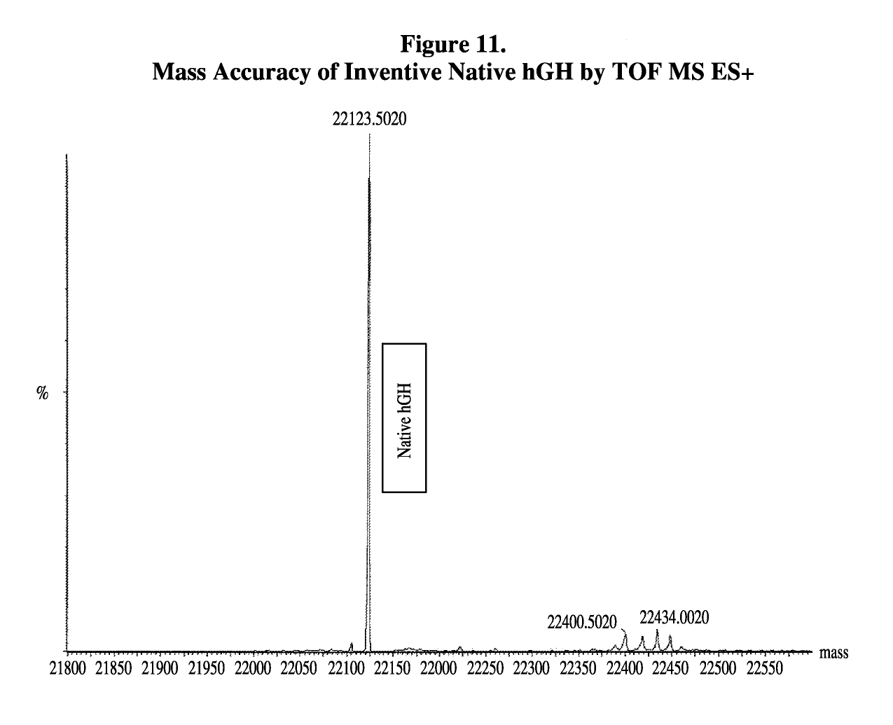

Peptide mapping was done on both Norditropin® and the present preparations of hGH after they were digested with trypsin. FIGS. 10 and 11 show the peptide maps.

In both cases, the maps were essentially identical. FIG. 10 has the various peptides identified. The peptide sequence of coverage of 69% for inventive growth hormone could be detected using MALDI-TOF (see Table 7).

TABLE 7
Sequence Coverage and Peptide Masses of Inventive hGH

Sequence Coverage: 69%
Matched Peptides shown in Bold:
MDFPTIPLSRLFDNAMLRAHRLHQLAFDTYQEFEEAYIPKEQKYSFLQNPQTSLCFSESIPTPS
NREETQQKSNLELLRISLLLIQSWLEPVQFLRSVFANSLVYGASDSNVYDLLKDLEEGIQTLMG
RLEDGSPRTGQIFKQTYSKFDTNSHNDDALLKNYGLLYCFRKDMDKVETFLRIVQCRSVEGSCG
F (SEQ ID NO: 4)

| Start-End | Observed | Mr (calc) | Miss | Sequence |
|---|---|---|---|---|
| 1-8 | 930.73 | 929.53 | 1 | FPTIPLSR (SEQ ID NO: 6) |
| 9-16 | 994.6 | 978.5 | 1 | LFDNAMLR (SEQ ID NO: 7) |
| | | | | Oxidation M |
| 17-19 | | 383.22 | 0 | AHR |
| 20-38 | 2342.08 | 2342.13 | 1 | LHQLAFDTYQEFEEAYIPK |
| | | | | (SEQ ID NO: 8) |
| 39-41 | | 404.21 | 0 | EQK |
| 42-64 | 2673.11 | 2673.26 | 1 | YSFLQNPQTSLCFSESIPTPSNR |
| | | | | (SEQ ID NO: 9) |
| 42-70 | 3416.79 | 3415.62 | 1 | YSFLQNPQTSLCFSESIPTPSNREETQQK |
| | | | | (SEQ ID NO: 10) |
| 71-77 | 844.68 | 844.49 | 1 | SNLELLR (SEQ ID NO: 11) |
| 78-94 | | 2055.20 | 0 | ISLLIQSWLEPVQFLR |
| | | | | (SEQ ID NO: 12) |
| 95-115 | | 2262.12 | 0 | SVFANSLVYGASDSNVYDLLK |
| | | | | (SEQ ID NO: 13) |
| 116-127 | 1361.82 | 1361.67 | 1 | DLEEGIQTLMGR (SEQ ID NO: 14) |
| 128-134 | 773.58 | 773.38 | 1 | LEDGSPR (SEQ ID NO: 15) |
| 135-140 | | 693.39 | 0 | TGQIFK (SEQ ID NO: 16) |
| 141-145 | | 626.31 | 0 | QTYSK (SEQ ID NO: 17) |
| 146-158 | 1489.80 | 1488.68 | | FDTNSHNDDALLK (SEQ ID NO: 18) |
| | | | | Desamido |
| 159-167 | 1205.58 | 1205.78 | 1 | NYGLLYCFR (SEQ ID NO: 19) |
| 169-178 | 1254.58 | 1253.64 | 1 | DMDKVETFLR (SEQ ID NO: 20) |
| 169-178 | 1269.76 | 1253.64 | 1 | DMDKVETFLR (SEQ ID NO: 21) |
| | | | | Oxidation M |
| 179-183 | | 675.36 | 0 | IVQCR (SEQ ID NO: 22) |
| 184-191 | 842.52 | 842.33 | 1 | SVEGSCGF (SEQ ID NO: 23) |

### Example 8

### In Vivo Study—Somatotropin and Humatrope®

The present study is provided to demonstrate the utility of the present hGH preparations in vivo. The present example demonstrates the utility to the described human growth hormone (somatotropin) preparations for providing long bone (tibia) growth comparable to that of conventional recombinant growth hormone preparations, particularly Humatrope®.

The somatotropin preparations were prepared as described herein. Humatrope® was obtained from commercial sources and used according to manufacturers directions.

Body Weight, Body Weight Change, and Percent Body Weight Change

Males: During the study phase, as expected with this previously established hypophysectomized rat model, the control group (0 μg/kg) treated with the vehicle showed a lack of body weight increase, in contrast to a significantly increase (90.2 g, 55.94%) observed with the Sham Surgery group. After the 14-day treatment, Somatropin at 25, 75, 250, and 500 μg/kg dose dependently increased group mean body weights by 5.2 g (5.36%), 18.2 g (18.82%), 30.6 g (31.49%), and 36.9 g (38.35%), respectively. During the same period, Humatrope® at 25, 75, 250, and 500 μg/kg also dose-dependently increased group mean body weights by 4.9 g (5.09%), 16.9 g (17.68%), 30.5 g (31.65%), and 36.2 g (37.82%), respectively. Statistical analysis indicated that, at the same respective dose levels, Somatropin was indistinguishable from Humatrope® in increasing the body weights.

With regard to the above treatment effects over time, at high doses, 250 and 500 μg/kg, both Somatropin and Humatrope® began to show a significant effect on body weight gain at Day 2. In comparison, at low doses, 25 and 75 μg/kg, Somatropin and Humatrope® began to show a significant body weight gain at Day 5 and Day 4, respectively. A fully dose dependent body weight gain was observed to be statistically significant by Day 9 for Somatropin and by Day 11 for Humatrope®. However, no significant differences in efficacy between Somatropin and Humatrope® were observed at the matching does levels during the entire treatment period.

Females: Consistent with the hypophysectomized rat model, the control group (0 μg/kg) treated with the vehicle had a limited body weight increase (4.4 g, 4.19%), in contrast to a significantly higher increase (45.8 g, 32.10%) with the Sham Surgery group during the study. In the same period, Somatropin at 25, 75, 250, and 500 μg/kg dose-dependently increased group mean body weights by 9.1 g (8.78%), 16.4 g (16.00%), 31.3 g (30.62%), and 36.0 g (34.97%), respectively. Humatrope® at 25, 75, 250, and 500 μg/kg also dose-dependently increased group mean body weights by 7.7 g (7.84%), 17.3 g (16.95%), 30.8 g (30.07%), and 45.8 g (40.44%), respectively. Statistical analysis indicated that, at 25, 75, and 250 μg/kg, Somatropin was indistinguishable from Humatrope® in increasing the body weights at the matching dose levels. However, at 500 μg/kg, Humatrope® caused a higher body weight gain than Somatropin.

With regard to the above treatment effects over time, at the highest dose (500 μg/kg), Somatropin and Humatrope® began to show a significant effect on body weight gain at Day 2 and Day 1, respectively. At 250 μg/kg, Somatropin and Humatrope® began to show a statistically significant effect on body weight gain at Day 3 and Day 2, respectively. In comparison, at low doses, 25 and 75 μg/kg, both Somatropin and Humatrope® began to produce a statistically significant body weight gain at Day 5 and Day 4, respectively. A fully dose-dependent body weight gain was observed to be statistically significant starting from Day 7 for Somatropin and Day 6 for Humatrope®. No significant differences in efficacy and potency between Somatropin and Humatrope® at the matching dose levels were observed in a daily manner during the entire treatment period except that at Days 14 and 15, animals treated with Humatrope® had body weights significantly higher than those treated with Somatropin at a dose level of 500 μg/kg. However, the analysis of the terminal body weight during necropsy at Day 15, this difference in body weight did not achieve a statistical significance.

Food Consumption: Due to the very low food consumption in hypophysectomized rats in general, calculation of daily food consumption based on weekly consumption was used to increase the sensitivity of data analysis.

Males: Consistent with the hypophysectomized rat model, at Weeks 1 and 2, Sham Surgery animals showed daily food consumption of 14.09 and 16.14 g, respectively, whereas the vehicle treated hypophysectomized animals (0 μg/kg) had much lower food consumption rates of 4.05 and 4.11 g/day, respectively.

At Week 1, animals treated with Somatropin at 25, 75, 250, and 500 μg/kg had food consumption rates of 4.54, 5.73, 5.23 and 4.84 g/day, respectively, and those treated with Humatrope® at 25, 75, 250, and 500 μg/kg, 4.02, 4.71, 5.08, and 4.83 g/day, respectively. There were no statistically significant and dose-related changes.

At Week 2, groups treated with Somatropin at 25, 75, 250, and 500 μg/kg had food consumption rates of 4.31, 4.90, 6.09, and 6.16 g/day, respectively, while those treated with Humatrope® at 25, 75, 250, and 500 μg/kg, 4.20, 4.89, 5.67, and 6.07 g/day, respectively. The increased food consumptions in the 75, 250, and 500 μg/kg groups were statistically significant for both Somatropin and Humatrope® as compared to the vehicle control. The increases in the 250 and 500 μg/kg groups were statistically significantly higher than those in the 75 μg/kg group for both Somatropin and Humatrope®. There were no significant differences in these effects between Somatropin and Humatrope® at the matching dose levels.

With regard to these effects on food consumption over time, a statistically significant and durable increase began at Day 7 and Day 9 for Somatropin and Humatrope® at 500 μg/day, respectively. At 250 μg/kg, such effect was observed starting from Day 10 for both Somatropin and Humatrope®. At each of these two dose levels, no statistically significant differences were observed between the two effectors.

Females: Consistent with the hypophysectomized rat model, at Weeks 1 and 2, Sham Surgery animals showed daily food consumption of 10.23 and 11.00 g, respectively, whereas the vehicle treated hypophysectomized animals had much lower food consumption rates of 5.97 and 5.62 g/day, respectively.

At Week 1, animals treated with Somatropin at 25, 75, 250, and 500 μg/kg had food consumption rates of 6.24, 5.33, 6.22, and 6.17 g/day, respectively, and those treated with Humatrope® at 25, 75, 250, and 500 μg/kg, 5.29, 5.64, 5.87, and 6.10 g/day, respectively. There were no statistically significant and dose-related changes.

At Week 2, groups treated with Somatropin at 25, 75, 250, and 500 μg/kg had food consumption rates of 5.69, 5.84, 6.44, and 7.13 g/day, respectively while those treated with Humatrope® at 25, 75, 250, and 500 μg/kg, 5.56, 6.29, 6.80, and 7.11 g/day, respectively. The increased food consumptions in the 250 and 500 μg/kg groups were statistically significant for both Somatropin and Humatrope® as compared to the vehicle control. There were no statistically significant differences between these two effectors at each of these two dose levels and between these two high dose levels.

With regard to the above effects over time, statistically significant and continuously higher food consumptions were observed starting from Day 7 for both Somatropin and Humatrope® at 500 μg/day compared to the vehicle control (0 μg/kg), except that at Day 13, the Somatropin-induced increase in food consumption did not reach statistical significance. At this highest dose level, no significant differences were observed between these two effectors.

Postmortem Study Evaluations: The pathology interpretation of the results is included in the results presented below.

Tibial Length

Males: For the left leg, consistent with the hypophysectomized rat model, the vehicle-treated control group (0 μg/kg) had a significantly shorter tibial length (3.03 cm) than that of the Sham Surgery group (3.52 cm). Groups treated with Somatropin at 25, 75, 250, and 500 μg/kg had tibial lengths of 3.04, 3.07, 3.18, and 3.15 cm, respectively, while those treated with Humatrope® at 25, 75, 250, and 500 μg/kg, 3.01, 3.09, 3.18, and 3.17 cm, respectively. At 250 and 500 μg/kg, Somatropin and Humatrope® statistically significantly increased the tibial length and no significant differences between the two doses and between these two effectors at the matching dose levels were observed.

For the right leg, consistent with the hypophysectomized rat model, the vehicle-treated control group (0 μg/kg) had a significantly shorter tibial length (2.98 cm) than that of the Sham Surgery group (3.43 cm). Groups treated with Somatropin at 25, 75, 250, and 500 μg/kg had tibial lengths of 3.01, 3.13, 3.17, and 3.24 cm, respectively, while those treated with Humatrope® at 25, 75, 250, and 500 μg/kg, 2.99, 3.09, 3.19, and 3.19 cm, respectively. At 75, 250 and 500 μg/kg, both Somatropin and Humatrope® statistically significantly increased the tibial length and no significant differences between the two high doses and between these two effectors at the matching dose levels were observed.

Females: For the left leg, consistent with the hypophysectomized rat model, the vehicle-treated control group (0 μg/kg) had a significantly shorter tibial length (3.14 cm) than that of the Sham Surgery group (3.40 cm). Groups treated with Somatropin at 25, 75, 250, and 500 μg/kg had tibial lengths of 3.09, 3.13, 3.17, and 3.21 cm, respectively, while those treated with Humatrope® at 25, 75, 250, and 500 μg/kg, 3.08, 3.13, 3.19, and 3.20 cm, respectively. At 250 and 500 μg/kg, Somatropin and Humatrope® increased the tibial length without achieving statistical significance.

For the right leg, consistent with the hypophysectomized rat model, the vehicle-treated control group (0 μg/kg) had a significantly shorter tibial length (3.14 cm) than that of the Sham Surgery group (3.40 cm). Groups treated with Somatropin at 25, 75, 250, and 500 μg/kg had tibial lengths of 3.08, 3.12, 3.19, and 3.21 cm, respectively, while those treated with Humatrope® at 25, 75, 250, and 500 μg/kg, 3.08, 3.13, 3.16, and 3.21 cm, respectively. At 250 and 500 μg/kg, Somatropin and Humatrope® increased the tibial length with a statistical significance observed for 500 μg/kg Humatrope® only. There were no statistically significant differences between these two high doses within each effector and between these two effectors at the matching dose levels.

Proximal Tibial Growth Plate Width

Males: For the left leg, consistent with the hypophysectomized rat model, the vehicle-treated control group (0 μg/kg) had a significantly shorter growth plate width (199 μm) than that of the Sham Surgery group (424 μm). Groups treated with Somatropin at 25, 75, 250, and 500 μg/kg had tibial growth plate widths of 234, 309, 414, and 474 μm, respectively, while those treated with Humatrope® at 25, 75, 250, and 500 μg/kg, 199, 303, 445, and 444 μm, respectively. At 75, 250 and 500 μg/kg, Somatropin and Humatrope® statistically significantly increased the growth plate width in a dose-dependent manner and no significant differences between these two effectors were observed.

For the right leg, consistent with the hypophysectomized rat model, the vehicle-treated control group (0 μg/kg) had a significantly shorter tibial length (204 μm) than that of the Sham Surgery group (445 μm). Groups treated with Somatropin at 25, 75, 250, and 500 μg/kg had tibial growth plate widths of 227, 322, 410, and 456 μm, respectively, while those treated with Humatropes at 25, 75, 250, and 500 μg/kg, 201, 306, 409, and 439 μm, respectively. At 75, 250 and 500 μg/kg, Somatropin and Humatrope® statistically significantly increased the growth plate in a dose-dependent manner and no significant differences between these two effectors were observed at the matching dose levels.

Females: For the left leg, consistent with the hypophysectomized rat model, the vehicle-treated control group (0 μg/kg) had a significantly shorter growth plate width (196 μm) than that of the Sham Surgery group (314 μm). Groups treated with Somatropin at 25, 75, 250, and 500 μg/kg had tibial growth plate widths of 212, 253, 377, and 382 μm, respectively, while those treated with Humatrope® at 25, 75, 250, and 500 μg/kg, 192, 252, 340, and 393 μm, respectively. At 75, 250 and 500 μg/kg, Somatropin and Humatrope® statistically significantly increased the growth plate width in a dose-dependent manner and there were no significant differences between these two effectors.

For the right leg, consistent with the hypophysectomized rat model, the vehicle-treated control group (0 μg/kg) had a significantly shorter tibial length (204 μm) than that of the Sham Surgery group (312 μm). Groups treated with Somatropin at 25, 75, 250, and 500 μg/kg had tibial growth plate widths of 208, 253, 380, and 387 μm, respectively, while those treated with Humatropes at 25, 75, 250, and 500 μg/kg, 192, 260, 336, and 380 μm, respectively. At 75, 250 and 500 μg/kg, Somatropin and Humatrope® statistically significantly increased the growth plate in a dose-dependent manner without significant differences being between these two effectors, except that, at 250 μg/kg, Somatropin was significantly more effective than Humatrope®.

Macroscopic: Macroscopic observations were noted in the pituitary gland of three animals at the time of terminal necropsy. One male treated with Humatrope® at 500 μg/kg was noted to have a portion of the pituitary present, as was one male treated with Somatropin at 500 μg/kg in which there was a 0.1 cm in diameter white nodule noted. One female treated with Somatropin at 75 μg/kg had tissue present at the pituitary site with the additional comment that the tissue could be the result of hematoma formation.

Organ Weights: There was a dose related increase in body weight in both males and females with increasing dose of administration of Somatropin and Humatrope® compared to the 0 μg/kg vehicle controls. A summary of the body weight changes is presented in the table 8.

TABLE 8
Treatment Related Changes in Body Weight Percent relative
to 0 μg/kg Vehicle Controls/Males and Females

| Treatment | Somatropin | | | | Humatrope ® | | | | Sham Surgery |
|---|---|---|---|---|---|---|---|---|---|
| Dose Level (μg/kg) | 25 | 75 | 250 | 500 | 25 | 75 | 250 | 500 | NA |
| Number Examined | 10 | 10 | 10 | 10 | 10 | 10 | 10 | 10 | 10 |
| Males | ↑ 5.4 | ↑ 18.4 | ↑ 32.7 | ↑ 37.3 | ↑ 3.3 | ↑ 15.6 | ↑ 30.7 | ↑ 37.9 | ↑ 162.3 |
| Females | ↑ 3.6 | ↑ 9.4 | ↑ 23.6 | ↑ 28.5 | ↑ 1.0 | ↑ 10.2 | ↑ 22.5 | ↑ 32.0 | ↑ 75.6 |

NA—not applicable;
↑—Increased

Absolute brain weights were minimally and similarly increased compared to the 0 μg/kg vehicle controls in males treated with both Somatropin and Humatrope® at 75, 250 and 500 μg/kg. In females, absolute brain weights were minimally and similarly increased in Somatropin-treated animals at 250 and 500 μg/kg and Humatrope®-treated animals at 75, 250 and 500 μg/kg compared to the 0 μg/kg vehicle controls. A summary of the absolute brain weight changes is presented in the table 9.

TABLE 9
Treatment Related Changes in Absolute Brain Weight Percent
relative to 0 μg/kg Vehicle Controls/Males and Females

| Treatment | Somatropin | | | | Humatrope ® | | | | Sham Surgery |
|---|---|---|---|---|---|---|---|---|---|
| Dose Level (μg/kg) | 25 | 75 | 250 | 500 | 25 | 75 | 250 | 500 | NA |
| Number Examined | 10 | 10 | 10 | 10 | 10 | 10 | 10 | 10 | 10 |
| Males | ↓ 1.9 | ↑ 3.3 | ↑ 3.6 | ↑ 3.3 | ↓ 2.3 | ↑ 2.7 | ↑ 3.1 | ↑ 4.1 | ↑ 8.8 |
| Females | ↑ 0.2 | 0.0 | ↑ 3.6 | ↑ 3.9 | ↓ 1.5 | ↑ 2.8 | ↑ 3.9 | ↑ 1.7 | ↑ 6.0 |

NA—not applicable;
↑—Increased;
↓—Decreased

Absolute heart weights were increased compared to the 0 μg/kg vehicle controls in all Somatropin-treated males, and in the Somatropin-treated females and Humatrope®-treated males and females at 75, 250 and 500 μg/kg. The increase in absolute heart weight in the females treated with Humatrope® at 75 μg/kg was minimal. A summary of the absolute heart weight changes is presented in the table 10.

TABLE 10
Treatment Related Changes in Absolute Heart Weight Percent
relative to 0 μg/kg Vehicle Controls/Males and Females

| Treatment | Somatropin | | | | Humatrope ® | | | | Sham Surgery |
|---|---|---|---|---|---|---|---|---|---|
| Dose Level (μg/kg) | 25 | 75 | 250 | 500 | 25 | 75 | 250 | 500 | NA |
| Number Examined | 10 | 10 | 10 | 10 | 10 | 10 | 10 | 10 | 10 |
| Males | ↑ 16.2 | ↑ 16.3 | ↑ 29.5 | ↑ 24.8 | 0.0 | ↑ 21.2 | ↑ 23.0 | ↑ 23.9 | ↑ 178.7 |
| Females | ↑ 0.6 | ↑ 12.5 | ↑ 14.5 | ↑ 22.5 | 0.0 | ↑ 2.6 | ↑ 12.5 | ↑ 20.5 | ↑ 102.3 |

NA—not applicable;
↑—Increased

There was a dose related increase in absolute kidney and liver weights compared to the 0 μg/kg vehicle controls in the Humatrope®-treated males and Somatropin-treated females. A similar trend was seen in the Somatropin-treated males with the exception of the 500 μg/kg Somatropin-treated males which had absolute kidney and liver weights slightly less than the absolute kidney and liver weights of the 250 μg/kg Somatropin-treated males. The Humatrope®-treated females at 25 μg/kg had an absolute kidney weight that was less than the 0 μg/kg vehicle controls; however the remaining dose levels exhibited a dose related increase in absolute kidney weights when compared to the 0 μg/kg vehicle controls. A summary of the absolute kidney and liver weight changes is presented in the tables below.

TABLE 11
Treatment Related Changes in Absolute Kidney Weight Percent
relative to 0 μg/kg Vehicle Controls/Males and Females

| Treatment | Somatropin | | | | Humatrope ® | | | | Sham Surgery |
|---|---|---|---|---|---|---|---|---|---|
| Dose Level (μg/kg) | 25 | 75 | 250 | 500 | 25 | 75 | 250 | 500 | NA |
| Number Examined | 10 | 10 | 10 | 10 | 10 | 10 | 10 | 10 | 10 |
| Males | ↑ 5.1 | ↑ 13.9 | ↑ 35.2 | ↑ 33.8 | ↑ 2.1 | ↑ 14.6 | ↑ 29.9 | ↑ 36.5 | ↑ 223.8 |
| Females | ↑ 1.7 | ↑ 13.5 | ↑ 20.3 | ↑ 26.8 | ↓ 11.9 | ↑ 6.4 | ↑ 23.7 | ↑ 32.6 | ↑ 127.4 |

NA—not applicable;
↑—Increased;
↓—Decreased

TABLE 12
Treatment Related Changes in Absolute Liver Weight Percent
relative to 0 μg/kg Vehicle Controls/Males and Females

| Treatment | Somatropin | | | | Humatrope ® | | | | Sham Surgery |
|---|---|---|---|---|---|---|---|---|---|
| Dose Level (μg/kg) | 25 | 75 | 250 | 500 | 25 | 75 | 250 | 500 | NA |
| Number Examined | 10 | 10 | 10 | 10 | 10 | 10 | 10 | 10 | 10 |
| Males | ↑ 9.3 | ↑ 19.3 | ↑ 35.3 | ↑ 33.2 | ↑ 6.2 | ↑ 17.5 | ↑ 24.6 | ↑ 29.8 | ↑ 226.6 |
| Females | ↑ 8.1 | ↑ 16.0 | ↑ 23.4 | ↑ 29.7 | ↑ 7.2 | ↑ 12.9 | ↑ 27.0 | ↑ 38.5 | ↑ 138.6 |

NA—not applicable;
↑—Increased

The brain weight was not dependent upon body weight as expected, and the weights of the heart, kidneys and liver were body weight dependent. There was no trend in relative organ weights to suggest any additional test article related alterations beyond increased size due to increased body weight.

In general, the Somatropin resulted in increases in organ weights such as heart, kidneys and liver, which are statistically indifferent from the equivalent Humatrope® dose and were correlated to body weight increases. Neither the Somatropin nor the Humatrope® treatment at any dose resulted in body and organ weights similar to that of the sham surgery control animals with the exception of brain weights which were only minimally and similarly increased in sham surgery animals compared to the hypophysectomized and treated animals.

Microscopic: Microscopic alterations were present affecting the growth plate in addition to the primary and secondary spongiosa and underlying trabecular bone which were consistent with the changes expected for this disease model. The lesions included chondrodystrophy of the growth plate characterized by a decreased thickness both of the proliferative and hypertrophic zones with maintenance of normal architecture of the zones in linear arrays; decreased ossification characterized by varying degrees of decreased or absent primary and secondary spongiosa and trabecular bone; and decreased osteoclasts present both at the interface of the growth plate.

It was evident that the hypophysectomy procedure to create the disease model was successful as the tibia lesions were most severe within the 0 μg/kg vehicle control rats. It was also evident that there was a dose dependent resolution of the disease state with both the Somatropin and Humatrope®-treatments.

At the higher doses of treatment for both the Somatropin and Humatrope® treatments, occasional rats had increased growth plate thickness characterized by a increased thickness both of the proliferative and hypertrophic zones with maintenance of normal architecture of the zones in linear arrays.

Males: Within the 0 μg/kg vehicle male controls, there was minimal to moderate chondrodystrophy, a mild to moderate decrease in ossification and a minimal to moderate decrease in osteoclasts. These lesions were of similar severity in the left and right tibias but were not always of equal severity with the left and right tibias within the same animal.

The microscopic alterations in the Somatropin-treated males at 25 μg/kg were similar to the 0 μg/kg vehicle controls with partial resolution of the decreased osteoclast number. The Humatrope®-treated males at 25 μg/kg had a slight decrease in resolution of the decreased osteoclast number when compared to the Somatropin-treated animals.

TABLE 13
Comparison of Microscopic Alterations in Somatropin and
Humatrope ®-Treated Rats/Males at 25 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 10 | 10 | 10 | 10 | 10 |
| Chondro- | 10 | 10 | 10 | 10 | 10 | 10 |
| dystrophy | | | | | | |
| minimal | 2 | 2 | 2 | 2 | 2 | 1 |
| mild | 7 | 7 | 8 | 6 | 7 | 9 |
| moderate | 1 | 1 | 0 | 2 | 1 | 0 |
| Decreased, | 10 | 10 | 10 | 10 | 10 | 10 |
| ossification | | | | | | |
| minimal | 0 | 0 | 0 | 0 | 0 | 0 |
| mild | 4 | 5 | 3 | 5 | 5 | 2 |
| moderate | 6 | 5 | 7 | 5 | 5 | 8 |
| Osteoclasts, | 10 | 10 | 10 | 10 | 10 | 10 |
| decreased | | | | | | |
| minimal | 2 | 2 | 1 | 2 | 2 | 1 |
| mild | 3 | 7 | 6 | 2 | 8 | 7 |
| moderate | 5 | 1 | 3 | 6 | 0 | 2 |

Within Somatropin and Humatrope®-treated males at 75 μg/kg, there was increased resolution of the lesions when compared to the 25 μg/kg dose groups. The Humatrope®-treated males at 75 μg/kg had comparable lesion resolution to the Somatropin-treated animals at this dose level; however, the Somatropin-treated males had increased resolution of both the decreased osteoclasts and decreased ossification lesions.

TABLE 14
Comparison of Microscopic Alterations in Somatropin and
Humatrope ®-Treated Rats/Males at 75 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 10 | 10 | 10 | 10 | 10 |
| Chondro- | 10 | 10 | 10 | 10 | 10 | 10 |
| dystrophy | | | | | | |
| minimal | 2 | 9 | 10 | 2 | 9 | 10 |
| mild | 7 | 1 | 0 | 6 | 1 | 0 |
| moderate | 1 | 0 | 0 | 2 | 0 | 0 |
| Decreased, | 10 | 10 | 10 | 10 | 10 | 10 |
| ossification | | | | | | |
| minimal | 0 | 3 | 0 | 0 | 3 | 0 |
| mild | 4 | 7 | 10 | 5 | 7 | 9 |
| moderate | 6 | 0 | 0 | 5 | 0 | 1 |
| Osteoclasts, | 10 | 8 | 10 | 10 | 6 | 8 |
| decreased | | | | | | |
| minimal | 2 | 7 | 8 | 2 | 5 | 7 |
| mild | 3 | 1 | 2 | 2 | 1 | 1 |
| moderate | 5 | 0 | 0 | 6 | 0 | 0 |

At 250 μg/kg, there was a considerable increase in resolution of all lesions in all treated males compared to the lower dose levels. There was increased resolution of all lesions in the Somatropin-treated animals when compared to the Humatrope®-treated males at this dose level. Within four Humatrope®-treated males, there was minimal thickening of the growth plate of the left tibia when compared to the sham surgery controls.

TABLE 15
Comparison of Microscopic Alterations in Somatropin and
Humatrope ®-Treated Rats/Males at 250 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 9 | 10 | 10 | 10 | 10 |
| Chondro- | 10 | 1 | 2 | 10 | 1 | 3 |
| dystrophy | | | | | | |
| minimal | 2 | 1 | 2 | 2 | 1 | 3 |
| mild | 7 | 0 | 0 | 6 | 0 | 0 |
| moderate | 1 | 0 | 0 | 2 | 0 | 0 |
| Decreased, | 10 | 3 | 7 | 10 | 4 | 9 |
| ossification | | | | | | |
| minimal | 0 | 2 | 5 | 0 | 4 | 8 |
| mild | 4 | 1 | 2 | 5 | 0 | 1 |
| moderate | 6 | 0 | 0 | 5 | 0 | 0 |
| Increased | 0 | 0 | 4 | 0 | 0 | 0 |
| thickness, | | | | | | |
| growth plate | | | | | | |
| minimal | 0 | 0 | 4 | 0 | 0 | 0 |
| Osteoclasts, | 10 | 2 | 3 | 10 | 1 | 2 |
| decreased | | | | | | |
| minimal | 2 | 2 | 3 | 2 | 1 | 2 |
| mild | 3 | 0 | 0 | 2 | 0 | 0 |
| moderate | 5 | 0 | 0 | 6 | 0 | 0 |

Within both the Somatropin and Humatrope®-treated males at 500 μg/kg, there was complete resolution of the chondrodystrophy; however in four or five males in each treatment group there was minimal thickening of the growth plate beyond that of the sham surgery control males. Findings were similar in these treatment groups except that there were fewer animals with decreased osteoclasts within the Somatropin-treated males.

TABLE 16
Comparison of Microscopic Alterations in Somatropin and
Humatrope ®-Treated Rats/Males at 500 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 10 | 10 | 10 | 10 | 10 |
| Chondro- | 10 | 0 | 1 | 10 | 0 | 0 |
| dystrophy | | | | | | |
| minimal | 2 | 0 | 1 | 2 | 0 | 0 |
| mild | 7 | 0 | 0 | 6 | 0 | 0 |
| moderate | 1 | 0 | 0 | 2 | 0 | 0 |
| Decreased, | 10 | 6 | 5 | 10 | 5 | 5 |
| ossification | | | | | | |
| minimal | 0 | 4 | 4 | 0 | 5 | 4 |
| mild | 4 | 2 | 1 | 5 | 0 | 1 |
| moderate | 6 | 0 | 0 | 5 | 0 | 0 |
| Increased | 0 | 4 | 5 | 0 | 5 | 2 |
| thickness, | | | | | | |
| growth plate | | | | | | |
| minimal | 0 | 4 | 5 | 0 | 5 | 2 |
| Osteoclasts, | 10 | 1 | 4 | 10 | 1 | 4 |
| decreased | | | | | | |
| minimal | 2 | 1 | 4 | 2 | 1 | 4 |
| mild | 3 | 0 | 0 | 2 | 0 | 0 |
| moderate | 5 | 0 | 0 | 6 | 0 | 0 |

Females: Within the 0 μg/kg vehicle female controls, there was mild to moderate chondrodystrophy, a minimal to moderate in decrease in ossification and a minimal to moderate decrease in osteoclasts. These lesions were of similar severity in the left and right tibias but were not always of equal severity with the left and right tibias within the same animal.

There was partial resolution of all microscopic alterations of similar extent in both the Somatropin and Humatrope®-treated females at 25 μg/kg compared to the 0 μg/kg vehicle controls.

TABLE 17
Comparison of Microscopic Alterations in Somatropin and
Humatrope ®-Treated Rats/Females at 25 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 10 | 10 | 10 | 10 | 10 |
| Chondro- | 10 | 10 | 10 | 10 | 10 | 10 |
| dystrophy | | | | | | |
| minimal | 0 | 0 | 1 | 0 | 0 | 1 |
| mild | 7 | 10 | 9 | 7 | 10 | 9 |
| moderate | 3 | 0 | 0 | 3 | 0 | 0 |
| Decreased, | 10 | 10 | 10 | 10 | 10 | 10 |
| ossification | | | | | | |
| minimal | 0 | 2 | 1 | 1 | 1 | 2 |
| mild | 8 | 7 | 4 | 6 | 9 | 4 |
| moderate | 2 | 1 | 5 | 3 | 0 | 4 |
| Osteoclasts, | 10 | 10 | 10 | 10 | 10 | 10 |
| decreased | | | | | | |
| minimal | 1 | 2 | 2 | 2 | 0 | 1 |
| mild | 4 | 7 | 6 | 3 | 9 | 6 |
| moderate | 5 | 1 | 2 | 5 | 1 | 3 |

Within Somatropin and Humatrope®-treated females at 75 μg/kg, there was increased resolution of the lesions when compared to the 25 μg/kg dose groups. The Humatrope®-treated females at 75 μg/kg had comparable lesion resolution to the Somatropin-treated animals at this dose level; however, the Humatrope®-treated females had increased resolution of the decreased osteoclast lesion in both tibias and of the chondrodystrophy in the right tibia only.

TABLE 18
Comparison of Microscopic Alterations in Somatropin and
Humatrope ®-Treated Rats/Females at 75 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 10 | 10 | 10 | 10 | 10 |
| Chondro- | 10 | 10 | 10 | 10 | 10 | 10 |
| dystrophy | | | | | | |
| minimal | 0 | 7 | 7 | 0 | 5 | 9 |
| mild | 7 | 3 | 3 | 7 | 5 | 1 |
| moderate | 3 | 0 | 0 | 3 | 0 | 0 |
| Decreased, | 10 | 10 | 10 | 10 | 10 | 10 |
| ossification | | | | | | |
| minimal | 0 | 2 | 2 | 1 | 3 | 2 |
| mild | 8 | 8 | 8 | 6 | 7 | 8 |
| moderate | 2 | 0 | 0 | 3 | 0 | 0 |
| Osteoclasts, | 10 | 10 | 9 | 10 | 10 | 9 |
| decreased | | | | | | |
| minimal | 1 | 3 | 6 | 2 | 3 | 7 |
| mild | 4 | 6 | 3 | 3 | 7 | 2 |
| moderate | 5 | 1 | 0 | 5 | 0 | 0 |

At 250 μg/kg, there was a considerable increase in resolution of all lesions in all treated animals. In general, there was increased resolution of all lesions in the Somatropin-treated animals when compared to the Humatropes-treated females at this dose level except for with the decreased osteoclast lesion in the right tibia.

TABLE 19
Comparison of Microscopic Alterations in Somatropin and
Humatrope ®-Treated Rats/Females at 250 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 10 | 10 | 10 | 10 | 10 |
| Chondro- | 10 | 0 | 1 | 10 | 0 | 1 |
| dystrophy | | | | | | |
| minimal | 0 | 0 | 1 | 0 | 0 | 1 |
| mild | 7 | 0 | 0 | 7 | 0 | 0 |
| moderate | 3 | 0 | 0 | 3 | 0 | 0 |
| Decreased, | 10 | 10 | 10 | 10 | 10 | 10 |
| ossification | | | | | | |
| minimal | 0 | 7 | 2 | 1 | 7 | 2 |
| mild | 8 | 3 | 8 | 6 | 3 | 8 |
| moderate | 2 | 0 | 0 | 3 | 0 | 0 |
| Osteoclasts, | 10 | 3 | 6 | 10 | 3 | 4 |
| decreased | | | | | | |
| minimal | 1 | 3 | 5 | 2 | 2 | 4 |
| mild | 4 | 0 | 1 | 3 | 1 | 0 |
| moderate | 5 | 0 | 0 | 5 | 0 | 0 |

Within both the Somatropin and Humatrope®-treated females at 500 μg/kg, there was nearly complete resolution of the chondrodystrophy; however in approximately half of the females in the Humatrope® treatment group there was minimal thickening of the growth plate beyond that of the sham surgery control females. Findings were similar in these treatment groups except with the decreased ossification lesion in the right tibias in which there was increased resolution of this lesion in the Somatropin-treated females compared to the Humatrope®-treated females.

TABLE 20
Comparison of Microscopic Alterations in Somatropin and
Humatrope ®-Treated Rats/Females at 500 μg/kg

| | Tibia | | | | | |
|---|---|---|---|---|---|---|
| | Left | | | Right | | |
| | Treatment | | | | | |
| | 0 μg/kg | Soma- | Huma- | 0 μg/kg | Soma- | Huma- |
| | Vehicle | tropin | trope ® | Vehicle | tropin | trope ® |
| | Number Examined | | | | | |
| | 10 | 10 | 10 | 10 | 10 | 10 |
| Chondro- | 10 | 0 | 0 | 10 | 1 | 0 |
| dystrophy | | | | | | |
| minimal | 0 | 0 | 0 | 0 | 1 | 0 |
| mild | 7 | 0 | 0 | 7 | 0 | 0 |
| moderate | 3 | 0 | 0 | 3 | 0 | 0 |
| Decreased, | 10 | 10 | 10 | 10 | 10 | 10 |
| ossification | | | | | | |
| minimal | 0 | 8 | 7 | 1 | 8 | 3 |
| mild | 8 | 2 | 3 | 6 | 2 | 7 |
| moderate | 2 | 0 | 0 | 3 | 0 | 0 |
| Increased | 0 | 0 | 6 | 0 | 0 | 5 |
| thickness, | | | | | | |
| growth plate | | | | | | |
| minimal | 0 | 0 | 6 | 0 | 0 | 5 |
| Osteoclasts, | 10 | 2 | 1 | 10 | 1 | 0 |
| decreased | | | | | | |
| minimal | 1 | 2 | 1 | 2 | 1 | 0 |
| mild | 4 | 0 | 0 | 3 | 0 | 0 |
| moderate | 5 | 0 | 0 | 5 | 0 | 0 |

Once daily subcutaneous administration of Somatropin or Humatrope® at 25, 75, 250, and 500 μg/kg for 14 days increased body weights, food consumption, tibia length, width of the proximal tibial growth plate, and resolution of growth hormone deficiency-associated abnormalities in bone tissues, such as chondrodystrophy and decreased ossification and osteoclasts, in both male and female hypophysectomized rats as compared to those treated with the vehicle control.

In general, the Somatropin resulted in increases in organ weights such as heart, kidneys and liver, which are statistically indifferent from the equivalent Humatrope® dose and were correlated to body weight increases. Neither the Somatropin nor the Humatrope® treatment at any dose resulted in body and organ weights similar to that of the sham surgery control animals with the exception of brain weights which were only minimally and similarly increased in Sham Surgery animals compared to the hypophysectomized and treated animals.

There is an overall dose dependency in all the observed effects by Somatropin and Humatrope® with the increases in body weight, tibial length and width of the proximal tibial growth plate and the resolution of bone abnormalities being the most significant effects. While some statistically significant differences were observed between matching dose groups, there were no consistent evidences suggesting a difference in relative potency and efficacy between Somatropin and Humatrope®.

### Example 10

### hGH E. coli Master Cell Bank Characterization

The present example demonstrates the utility of the present invention for providing a stable transformed E. coli mater cell bank suitable for the commercial manufacture of high grade recombinant human growth hormone. The analysis was performed to establish the qualification of the cell bank as a cGMP quality cell bank stock suitable for producing clinical grade human recombinant growth hormone. Plasmid copy number analysis was performed by qPCR using Beckman Coulter Genomic assays ECOAPH v 1.0 (detects the kanamycin resistance gene from transposon.

The Master cell bank was further analyzed to identify specific characteristics that define the stably transformed E. coli cells that carry the Met-hGH containing plasmids. Some of the characteristics that may be used to define the transformed E. coli cells include plasmid copy number, DNA sequence analysis of isolated plasmids, genetic stability testing assessment, marker retention, cell viability count, and restriction mapping characterization. Plasmid DNA sequencing, plasmid copy number determination, and genetic stability testing assessment was conducted on transformed E. coli prepared according to these procedures provided in Example 2. The test results are summarized in Table 21.

TABLE 21

| Test | Results |
|---|---|
| Detection of Non-Host Organisms | Negative |
| in Microbial | |
| Phage Testing | Negative |
| Confirmation of Host System Identity- | Identity: E. coli (99.9%) |
| E. coli | |
| Plasmid Retention by Selective Marker | 100% Plasmid Retained |
| Sensitivity | |
| DNA sequencing | 582 bp sequence identical to |
| | reference sequence |
| Copy Plasmid number | 23.69 +/− 1.11 |
| Restriction Endonuclease Mapping | Restriction digestions of test |
| | article and reference plasmid |
| | yield identical patterns |
| Viable Cell Count determination | 3.1 × 10¹⁴ CFU/mL |

Eight (8) vials of the plasmid material from transformed E. coli cells were analyzed. These test articles were as identified in the study as noted in Table 22:

TABLE 22

| | | Test | Storage |
|---|---|---|---|
| Type | Identity | Designation | Condition |
| Cells | E03-NhGh (pTrcHis2AKan) Lot# | ZZ191094 | −80° C. |
| | EBI-0809-001 - Vial 03 | | |
| Cells | E03-NhGh (pTrcHis2AKan) Lot# | ZZ191091 | −80° C. |
| | EBI-0809-001 - Vial 19 | | |
| Cells | E03-NhGh (pTrcHis2AKan) Lot# | ZZ191095 | −80° C. |
| | EBI-0809-001 - Vial 28 | | |
| Cells | E03-NhGh (pTrcHis2AKan) Lot# | ZZ191092 | −80° C. |
| | EBI-0809-001 - Vial 44 | | |
| Cells | E03-NhGh (pTrcHis2AKan) Lot# | ZZ203664 | −80° C. |
| | EBI-0809-001 - Vial 45 | | |
| Cells | E03-NhGh (pTrcHis2AKan) Lot# | ZZ203663 | −80° C. |
| | EBI-0809-001 - Vial 47 | | |
| Cells | E03-NhGh (pTrcHis2AKan) Lot# | ZZ191093 | −80° C. |
| | EBI-0809-001 - Vial 57 | | |
| Cells | E03-NhGh (pTrcHis2AKan) Lot# | ZZ191096 | −80° C. |
| | EBI-0809-001 - Vial 66 | | |

The control articles used in the analysis were as noted in Table 23:

TABLE 23

| | | Test | Storage |
|---|---|---|---|
| Type | Identity | Designation | Condition |
| Host | BL21 Competent Cells - Novagen | ZZ191097 | −80° C. |
| Host | BL21 Competent Cells - Novagen | ZZ191098 | −80° C. |
| Plasmid | pTrcHis2Akan reference plasmid | ZZ191099 | −80° C. |
| Plasmid | pTrcHis2Akan reference plasmid | ZZ191100 | −80° C. |

DNA Sequence Analysis of Isolated Plasmids:

Regulatory commission grade double strand DNA sequence (2-fold coverage for each strand) was generated for the 582 bp plasmid insert of Master Cell Bank E03-NhGH. Plasmid DNA was isolated from an LB broth plus kanamycin culture grown from an aliquote of each test article. Plasmid DNA was prepared from each culture using a Qiagen QIAmp DNA Mini kit, then assessed by agarose gel electrophoresis and quantitated by spectrophotometry. The plasmid DNA was used as the template for DNA sequencing. The plasmid DNA was used as the template for DNA sequencing. The sequencing primers used are shown below:

TABLE 24

| Primer Name | Primer Sequence |
|---|---|
| NhGH F1_836-001F | ATCAGACAATCTGTGTGGGTCTG |
| | (SEQ ID NO: 28) |
| NhGH R1_836-001R | ATTCCGACACCGTCCAACCGTG |
| | (SEQ ID NO: 29) |
| NhGH F2_836-001F | ATTCCGACACCGTCCAACCGTG |
| | (SEQ ID NO: 30) |
| NhGH R2_836-001R | ATGATGGTCGACGGCGCTATTCAG |
| | (SEQ ID NO: 31) |

DNA sequencing was performed via the BigDye® Terminator Cycle Sequencing Kit (Applied Biosystems). Sequencing reactions were purified then analyzed on an ABI PRISM 3730×1 DNA Analyzer. The raw data was analyzed using Sequencing Analysis software (Applied Biosystems). Sequence data was assembled and analyzed using the Sequencer software (Gene Codes).

DNA Sequence Analysis of Isolated Plasmids:

PCR amplification of the test articles produced amplicons of the expected sizes for each primer set. No differences were observed in the derived consensus sequences generated for either test article and the reference sequences employed in this analysis.

Plasmid Copy Number Determination by qPCR:

Copy number analysis was performed by qPCR using the Beckman Coulter Genomics assays ECOAPH v1.0 (detects the kanamycin resistance gene from transposon Tn903) and ECODNAP v1.1. (detects the E. coli DNA polymerase gene). The ECODNAP v1.1 assay was used as an endogenous control to normalize for the number of cells assayed. A series of dilutions of the pTrcHis2AKan plasmid were used to generate a standard curve to calibrate the ECOAPH v1.0 target assay. Total DNA extracted from the host E. coli cells was used to generate a standard curve to calibrate the ECODNAP v1.1 assay. The assumptions were made that there is a single DNA polymerase gene.

Total DNA was extracted from each master cell bank (“MCB”) using the Promega Maxwell 16 robot. One target assay (ECOAPH v1.0 detecting the plasmid) and one normalizing assay (ECODNAP v1.1, detecting the E. coli genomic DNA) were performed on the extracted DNA from each MCB. Six independent dilutions of DNA from each MCB were prepared and analyzed in duplicate.

Each of the qPCR reactions was assembled based upon the TaqMan™ Universal PCR Master Mix protocol (Applied Biosystems). The reactions were run in duplicate. The reactions were thermal cycled using the following conditions: 50° C. for 2 minutes, 95° C. for 10 minutes, followed by 40 cycles of 95° C. for 15 seconds and 60° C. for 1 minute. Data was collected by the ABI Prism 7900™ Sequence Detection System software (Applied Biosystems). Copy number was calculated as the number of copies (target gene) per cell (normalizing gene).

Results: Plasmid Copy Number Determination by qPCR

TABLE 25

| Test Article | Copy Number |
|---|---|
| E03NhGH | 23.69 ± 1.11 |

Method: Bacterial Species Characterization: The Master Cell Bank samples were streaked on agar plates for colony isolation and incubated at 37° C. for approximately 16 hours. BL-21 Escherichia coli cells were processed in parallel to serve as a control. A single colony from each plate was transferred to a 0.85% solution, and the suspension used to inoculate API 20E kit test strips (bioMerieux) which are composed of 23 microtubes to perform 23 biochemical tests for the identification of glucose-fermenting Gram negative rods. The strips were incubated for 18-24 hours at 37° C. then scored to identify the genus and species of the bacterium. Gram staining was performed from colonies representing both test articles and the control cells then fixed to glass slides. Each group of cells was Gram stained and viewed under 100× magnification. E. coli cells were identified as rod shaped bacteria. Confirmation of the host control cells validated the assay and thus no repeat was necessary.

Results: Bacterial Species Characterization: E03-NhGH was identified to be Escherichia coli (99.9% ID). Gram stain results indicated the presence of gram negative cells.

Method: Cell Purity Assessment: Three vials were selected from the master cell bank (E03-NhGH vials 44, 57, and 66). Six 100 mm Tryptic Soy Agar plates were inoculated from each vial with 100 uL. Two additional plates were inoculated with PBS to serve as controls. Plates were incubated at 25° C. or 37° C. for 7 days and monitored daily for heterogeneous growth.

Results: Cell Purity Assessment: E03NhGH displayed completely homogeneous lawn growth. Not growth was detectable on either negative control plate inoculated with PBS.

Method: Phage Contamination Assessment: Supernatants were collected from both chloroform treated and non-treated MCB samples. The supernatants were plated with JM109 cells to test for plaque formation. Supernatants from K-12 and lambda phage were used as positive controls, and supernatant from phage-free XL1-Blue and lambda suspension medium were used as negative controls. Plates were all observed after 16 hours for plaque formation, and the number of plaques recorded.

Results: Phage Contamination Assessment: E03NhGH both displayed zero pfu/mL, indicated lack of detectable phage contamination.

Method: Viable Cell Count Determination: Viable cell counting was performed by preparing a series of dilutions from MCB E03NhGH samples and plating three aliquots of each dilution on separate 100 mm LB agar plus kanamycin plates. As a negative control, 100 μl of PBS was spread onto a 100 mm LB agar plus kanamycin plate. The plates were incubated at 37° C. for approximately 16 hours. After incubation, the number of colonies was counted on the plates where individual colonies were observed. The viable cell count per milliliter of sample was calculated.

Results: Viable Cell Count Determination:

TABLE 26
Viable Cell Count Results for E03-NhGH

| | 10⁻¹¹ Dilution | 10⁻¹² Dilution | 10⁻¹³ Dilution |
|---|---|---|---|
| Replica 1 | 320 | 200 | 165 |
| Replica 2 | 296 | 198 | 140 |
| Replica 3 | 342 | 202 | 139 |
| Average | 319.3 | 200 | 148 |
| Vol Plated | 100 | 100 | 100 |
| CFU/mL of Dilution | 3193 | 2000 | 1480 |

Method: Marker Retention: 320 colonies from each MCB were tested for the presence or absence of the selective marker (the kanamycin resistance gene on the plasmid). The sample and positive (kanamycin resistance) and negative (kanamycin sensitive) cells were plated onto LB agar to obtain isolated colonies. For each MCB, four master plates—each containing 80 sample colonies, 8 positive controls, and 8 negative controls—were created. Colonies from the master plates were then transferred to selective (LB agar plus kanamycin) and non-selective media (LB agar). Results are reported as the percentage of colonies retaining the kanamycin marker (those that grew on the selective medium).

Results: Marker Retention:

TABLE 27

| | # Colonies on | # Colonies on Non- | % Marker |
|---|---|---|---|
| Test Article | Selective Media | Selective Media | Retention |
| E03-NhGH | 320 | 320 | 100% |

Method: Restriction Mapping: Plasmid DNA isolated from an LB broth plus kanamycin culture grown from aliquots of each test article was restriction enzyme digested using the restriction enzymes listed in Table 28.

TABLE 28
E03-NhGH Digestions

| Restriction Enzyme | Expected Fragments (kb) |
|---|---|
| Nde I | ~6.3 |
| Pst I | ~6.3 |
| Sma I | ~6.3 |
| Xho I | ~6.3 |
| Nde I, EcoR I | ~5.7, ~0.6 |

Results: Restriction Mapping:

TABLE 29
E03-NhGH Digestions

| Restriction Enzyme | Observed Fragments (kb) |
|---|---|
| Nde I | 6.557 |
| Pst I | 6.557 |
| Sma I | 6.557 |
| Xho I | 6.557 |
| Nde I, EcoR I | 5.631, 0.603 |

### Example 11

### Characterization of hGH and Formulations

The present example demonstrates the utility of the present invention for providing higher purity preparations of hGH without the typically required additional purification (chromatography) steps characteristic of typical methods in common use to prepare recombinant hGH in commercial product (API). The present example also demonstrates the utility of the present invention for providing a formulation of the herein described API hGH.

Currently acceptable hGH API preparations include a related substances amount of not more than 6%, and a limitation of the amount of high molecular weight substances of not more than 4%. The present recombinant hGH produced according to the herein disclosed methods has been identified to have a related substances content of not more than 1.5%, and high molecular weight content species of not more than 0.6%.

In the present methods, an ion exchange chromatography step is used to separate the Met Asp hGH from native hGH. The extra Asp residue in Met Asp makes the pI of the molecule lower than native hGH. The decrease in pI makes the Met Asp hGH elute later in the gradient than native hGH. Using in-process analytical evaluation of the fractions one can determine how to pool the main peak hGH.

The reverse phase HPLC column is used to remove the desamido forms and multimeric forms of native hGH. The desamido forms will elute sooner than main peak hGH and the multimeric hGH will elute much later (more hydrophobic). Again, in process RP-HPLC can be use to pool the main peak hGH minimizing the desamido forms of hGH. The pool from RP-HPLC is made 20% acetonitrile to minimize the chance of any multimeric hGH forming.

In conventional methods of producing hGH, acetonitrile is used throughout to minimize multimeric forms of hGH, plus SEC chromatography at the end. SEC chromatography is expensive in production settings. These steps are effectively eliminated in the present methods.

A general rule of conventional production, additional steps add cost and also results in lower yield. Yield on average is usually reduced between (10-15%) per additional step, with a typically higher loss with additional chromatography steps (10-40%) added to a process.

In addition, the method of production of the present invention does not include a size exclusion chromatography step at the end of the process. This is a step typically employed in conventional hGH preparation that is avoided according to the present manufacturing process. In part, this is due to the lack of multimeric forms that are formed and/or otherwise present through the production methods disclosed herein.

Formulations of recombinant hGH: The following formulations may be prepared with the present API of recombinant hGH.

Diluent: 0.3% Metacresol and 17% Glycerin in Water for Injection

5 mg Vial of hGH, 5 mg of native Growth Hormone, 25 mg of Mannitol, 5 mg Glycine.

This material is mix to homogeneity and sterile filtered. The material is dispensed into vials and lyophilized. The material used in the animal study was formulated this way.

While in the foregoing specification this invention has been described in relation to certain preferred embodiments thereof, and many details have been set forth for the purpose of illustration, it will be apparent to those skilled in the art that the invention is subject to additional embodiments and that certain of the details described herein can be varied considerably without departing from the basic principles of the invention.

What is claimed is:

1. A method for preparing a composition comprising a recombinant human growth hormone preparation, said recombinant human growth hormone preparation having 2% or less of other than a peptide having an amino acid sequence of native recombinant human growth hormone, said method comprising the steps of:
preparing a vector comprising a nucleic acid sequence encoding native human growth hormone having a dipeptide met-asp appended to the N-terminus to provide a transformation vector;
transforming an E. coli with said transformation vector to provide a transformed E. coli;
inducing the transformed E. coli to transcribe the hGH-dipeptide met-asp sequence;
harvesting the induced transformed E. coli and collecting an enriched population of transformed E. coli comprising inclusion bodies;
solubilizing said inclusion bodies in a high urea preparation and collecting metasphGH;
refolding the metasphGH in the presence of a phosphate buffer by dialysis against a phosphate buffer solution;
cleaving the Met Asp-hGH to provide a recombinant hGH preparation;
processing said recombinant hGH preparation over an ion exchange chromatography column and collecting ion exchanged hGH pooled fractions;
subjecting the ion exchanged hGH pooled fraction to reverse phase chromatography to provide a RPHPLC pool; and
processing the RPHPLC pool to provide to provide a purified recombinant hGH.

2. The method of claim 1 wherein the processing of the RPHPLC pool comprises adjusting, desalting; and lyophilizing the RPHPLC pool to provide a purified recombinant hGH.

3. The method of claim 1 wherein the ion exchange chromatography step is conducted in the absence of acetonitrile.

4. The method of claim 1 wherein the E. coli is a BL21.

5. The method of claim 1 wherein cleaving the MetAsp-hGH is performed in the presence of an appropriate ratio of cathepsin C.

6. The method of claim 1 wherein the peptide having an amino acid sequence other than native human growth hormone is desamidated hGH.

7. The method of claim 1 wherein the recombinant human growth hormone is essentially free of dimer and multimer hGH species.

8. The method of claim 1 wherein the transformation vector is pTrcHis2A/kan-MDN hGH.

9. A recombinant human growth hormone preparation having 2% or less of a desamidated form of hGH as determined by reverse phase HPLC chromatogram.

10. The recombinant human growth hormone preparation of claim 8 comprising 1% or less of a desamidated form of hGH as determined by reverse phase HPLC chromatogram.

11. The recombinant human growth hormone preparation of claim 9 essentially free of a desamidated form of hGH as determined by reverse phase HPLC chromatogram.

12. Recombinant human growth hormone produced by a method which comprises:
culturing E. coli cells which contain a recombinant DNA molecule, which DNA molecule comprises a nucleotide sequence encoding human growth hormone preceded by a methionine-asparagine residue at its N-terminus, said encoding nucleotide sequence contained in an expression system effective in producing said encoded human growth hormone in said E. coli cells; and
recovering the human growth hormone from the culture.

13. The human growth hormone of claim 9 which is in purified and isolated form.

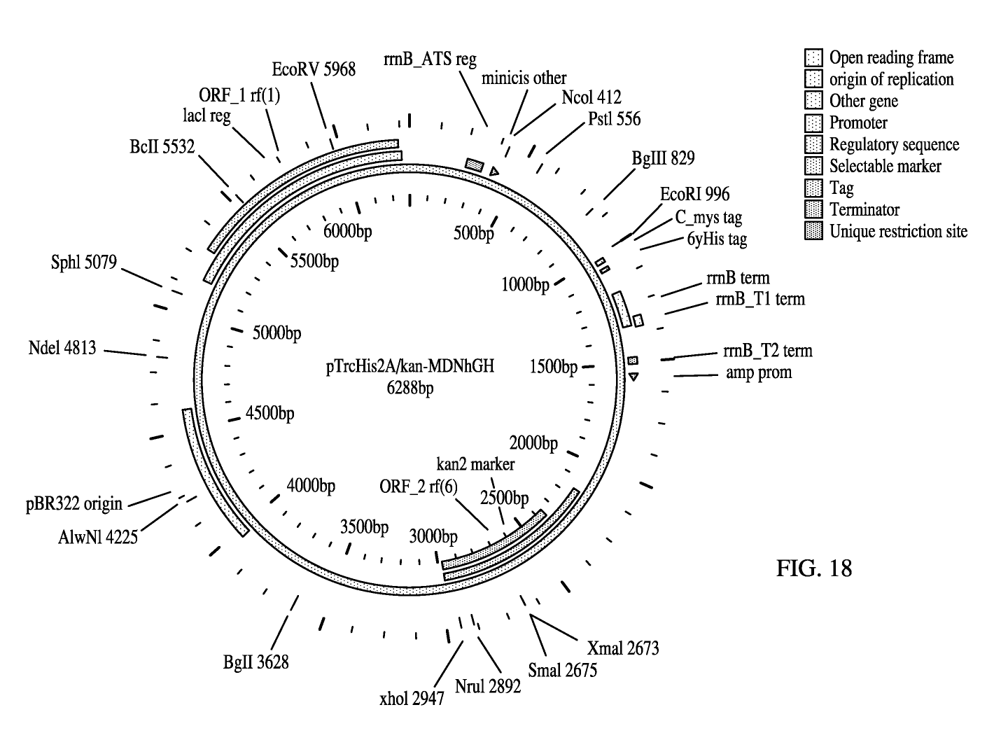

14. An active pharmaceutical ingredient (API) of recombinant human growth hormone isolated from a recombinant E. coli transformed with the transformation vector of FIG. 18.

15. The active pharmaceutical ingredient (API) of claim 14 formulated as an injectable preparation.

16. A transformed recombinant E. coli comprising a plasmid pTrcHis2A/kan-metasphGH.

17. The recombinant E. coli of claim 16 wherein the E. coli is a BL21 E. coli.

18. A Master Cell Bank comprising the transformed recombinant E. coli of claim 16.

19. A recombinant plasmid pTrcHis2A/KANNhGH as depicted in FIG. 18.


SEQUENCE LISTING (31 sequences)

SEQ ID NO: 1 - DNA, 5745 bp, Artificial Sequence
  Description of Artificial Sequence Synthetic polynucleotide
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 2 - DNA, 785 bp, Artificial Sequence
  Description of Artificial Sequence Synthetic polynucleotide
tcgaccggaattatcgattaactttattattaaaaattaaagaggtatatattaatgtatcgattaaataaggaggaataaaccatggacttcccaactatcccactgtctcgtctgttcgataacgctatgcttcgtgctcatcgtcttcatcagctggcctttgacacctaccaggagtttgaagaagcctatatcccaaaggaacagaagtattcattcctgcagaacccgcagacctccctctgtttctcagagtctattccgacccgtccaaccgtgaggaaacacaacagaaatccaacctggagctgctccgcatctccctgctgctcatccagtcgtggctggagccggtgcagttcctccgtagtgtcttcgccaacagcctggtgtacggcgcctctgacagcaacgtctatgacctcctgaaggacctggaggaaggcatccaaaccctgatgggtcgtctggaagatggcagcccgcgtactggtcagatcttcaagcagacctacagcaagttcgacacaaactcacacaacgatgacgcactgctcaagaactacggtctgctctactgcttccgtaaggacatggacaaggtcgagacattcctgcgcatcgtgcagtgccgctctgtggagggcagctgtggcttctaggaattcgaagcttgggcccgaacaaaaactcatctcagaagaggatctgaatagcgccgtcgaccatcatcatcatcatcattgagtttaaacggtctccagcttggctgttttggcgga

SEQ ID NO: 3 - DNA, 582 bp, Homo sapiens
atggacttcccaactatcccactgtctcgtctgttcgataacgctatgcttcgtgctcatcgtcttcatcagctggcctttgacacctaccaggagtttgaagaagcctatatcccaaaggaacagaagtattcattcctgcagaacccgcagacctccctctgtttctcagagtctattccgacaccgtccaaccgtgaggaaacacaacagaaatccaacctggagctgctccgcatctccctgctgctcatccagtcgtggctggagccggtgcagttcctccgtagtgtcttcgccaacagcctggtgtacggcgcctctgacagcaacgtctatgacctcctgaaggacctggaggaaggcatccaaaccctgatgggtcgtctggaagatggcagcccgcgtactggtcagatcttcaagcagacctacagcaagttcgacacaaactcacacaacgatgacgcactgctcaagaactacggtctgctctactgcttccgtaaggacatggacaaggtcgagacattcctgcgcatcgtgcagtgccgctctgtggagggcagctgtggcttctag

SEQ ID NO: 4 - protein, 193 aa, Homo sapiens
MDFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 5 - DNA, 6288 bp, Artificial Sequence
  Description of Artificial Sequence Synthetic polynucleotide
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggacttcccaactatcccactgtctcgtctgttcgataacgctatgcttcgtgctcatcgtcttcatcagctggcctttgacacctaccaggagtttgaagaagcctatatcccaaaggaacagaagtattcattcctgcagaacccgcagacctccctctgtttctcagagtctattccgacaccgtccaaccgtgaggaaacacaacagaaatccaacctggagctgctccgcatctccctgctgctcatccagtcgtggctggagccggtgcagttcctccgtagtgtcttcgccaacagcctggtgtacggcgcctctgacagcaacgtctatgacctcctgaaggacctggaggaaggcatccaaaccctgatgggtcgtctggaagatggcagcccgcgtactggtcagatcttcaagcagacctacagcaagttcgacacaaactcacacaacgatgacgcactgctcaagaactacggtctgctctactgcttccgtaaggacatggacaaggtcgagacattcctgcgcatcgtgcagtgccgctctgtggagggcagctgtggcttctaggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 6 - protein, 8 aa, Homo sapiens
FPTIPLSR

SEQ ID NO: 7 - protein, 8 aa, Homo sapiens
LFDNAMLR

SEQ ID NO: 8 - protein, 19 aa, Homo sapiens
LHQLAFDTYQEFEEAYIPK

SEQ ID NO: 9 - protein, 23 aa, Homo sapiens
YSFLQNPQTSLCFSESIPTPSNR

SEQ ID NO: 10 - protein, 29 aa, Homo sapiens
YSFLQNPQTSLCFSESIPTPSNREETQQK

SEQ ID NO: 11 - protein, 7 aa, Homo sapiens
SNLELLR

SEQ ID NO: 12 - protein, 16 aa, Homo sapiens
ISLLIQSWLEPVQFLR

SEQ ID NO: 13 - protein, 21 aa, Homo sapiens
SVFANSLVYGASDSNVYDLLK

SEQ ID NO: 14 - protein, 12 aa, Homo sapiens
DLEEGIQTLMGR

SEQ ID NO: 15 - protein, 7 aa, Homo sapiens
LEDGSPR

SEQ ID NO: 16 - protein, 6 aa, Homo sapiens
TGQIFK

SEQ ID NO: 17 - protein, 5 aa, Homo sapiens
QTYSK

SEQ ID NO: 18 - protein, 13 aa, Homo sapiens
FDTNSHNDDALLK

SEQ ID NO: 19 - protein, 9 aa, Homo sapiens
NYGLLYCFR

SEQ ID NO: 20 - protein, 10 aa, Homo sapiens
DMDKVETFLR

SEQ ID NO: 21 - protein, 10 aa, Homo sapiens
DMDKVETFLR

SEQ ID NO: 22 - protein, 5 aa, Homo sapiens
IVQCR

SEQ ID NO: 23 - protein, 8 aa, Homo sapiens
SVEGSCGF

SEQ ID NO: 24 - protein, 6 aa, Homo sapiens
VETFLR

SEQ ID NO: 25 - protein, 10 aa, Homo sapiens
NYGLLYCFRK

SEQ ID NO: 26 - protein, 16 aa, Homo sapiens
FPTIPLSRLFDNAMLR

SEQ ID NO: 27 - protein, 6 aa, Artificial Sequence
  Description of Artificial Sequence Synthetic 6xHis tag
HHHHHH

SEQ ID NO: 28 - DNA, 23 bp, Artificial Sequence
  Description of Artificial Sequence Synthetic primer
atcagacaatctgtgtgggtctg

SEQ ID NO: 29 - DNA, 22 bp, Artificial Sequence
  Description of Artificial Sequence Synthetic primer
attccgacaccgtccaaccgtg

SEQ ID NO: 30 - DNA, 22 bp, Artificial Sequence
  Description of Artificial Sequence Synthetic primer
attccgacaccgtccaaccgtg

SEQ ID NO: 31 - DNA, 24 bp, Artificial Sequence
  Description of Artificial Sequence Synthetic primer
atgatggtcgacggcgctattcag